(19) United States
(12) Patent Application Publication
(10) Pub. No.: US 2026/0347944 A1
(43) Pub. Date: Nov. 5, 2026

(54) METHODS AND SYSTEMS FOR DETECTION OF CANCER BASED ON DNA METHYLATION OF SPECIFIC CPG SITES

(71) Applicant: Foundation Medicine, Inc., Boston, MA (US)

(72) Inventors: Mark R. KENNEDY, Cambridge, MA (US); Ray Abrams GREENSTEIN, West Hartford, CT (US)

(73) Assignee: Foundation Medicine, Inc., Boston, MA (US)

(21) Appl. No.: 19/120,708

(22) PCT Filed: Oct. 12, 2023
(86) PCT No.: PCT/US2023/076666

§ 371 Date: Apr. 11, 2025

Related U.S. Application Data
(60) Provisional application No. 63/415,794, filed on Oct. 13, 2022.

(51) Int. Cl. C12Q 1/6886; C12Q 1/6858

(52) U.S. Cl. CPC C12Q 1/6886; C12Q 1/6858; C12Q 2600/154

(57) ABSTRACT

Methods for detecting disease in a subject are described. In some instances, the methods may comprise: receiving sequence read data for a plurality of sequence reads derived from a sample; determining a methylation fraction value for a plurality of CpG sites within each genomic region of a set of selected genomic regions based on the sequence read data; and performing a statistical analysis to compare a distribution of the methylation fraction values determined for a plurality of CpG sites within one or more selected genomic regions in the subject to a corresponding distribution of methylation fraction values determined for the plurality of CpG sites within one or more selected genomic regions for a plurality of healthy individuals, where a difference between the distribution of methylation fraction values determined for the subject and that determined for the plurality of healthy individuals is indicative of a presence of disease in the subject.

### CROSS-REFERENCE TO RELATED APPLICATIONS

This application claims the priority benefit of U.S. Provisional Patent Application Ser. No. 63/415,794, filed Oct. 13, 2022, the contents of which are incorporated herein by reference in their entirety.

### FIELD OF THE INVENTION

The present disclosure relates generally to methods and systems for analyzing genomic profiling data, and more specifically to methods and systems for the detection of disease (e.g., cancer) using genomic profiling data.

### BACKGROUND

DNA methylation has been shown to alter gene activity, and has been implicated in the regulation of stable and heritable patterns of gene expression. The methylation of nucleic acid bases (e.g., cytosines) located in a gene promoter region, for example, often suppresses gene transcription. In vertebrates, cytosine methylation occurs primarily at CG dinucleotide sequences that are referred to as CpG sites.

Changes in the methylation patterns for certain genes or genomic regions can be associated with certain aspects of a genetic disease, e.g., a cancer. For example, changes in methylation patterns may be indicative of the driving mechanism underlying a cancer. Detection of changes in DNA methylation status of certain genes or genomic regions thus provides a potential means to detect the presence of disease, e.g., cancer, and may be prognostic for patient response to certain therapies. Previous work has focused on identifying genomic loci or genomic regions that are differentially-methylated in samples from healthy and diseased subjects. However, variability in DNA methylation levels that arises during DNA replication and cell division in vivo, as well as noise in the in vitro techniques used to detect DNA methylation status, have limited the utility of this approach.

### BRIEF SUMMARY OF THE INVENTION

Disclosed herein are methods and systems for the detection of disease, e.g., cancer, based on DNA methylation status that comprise first defining a set of “compact” genomic regions that exhibit correlated methylation states in a specified cohort of individuals, e.g., healthy individuals, and then comparing the distribution of methylation status values determined for the same set of “compact” genomic regions in an individual subject to that for the cohort to detect perturbations in methylation status and determine the likelihood that a disease, e.g., cancer, is present in the subject. As described herein, a “compact” genomic region is a segment of the genome that includes a plurality of CpG sites in relatively close proximity and that exhibits relatively consistent and correlated levels of CpG methylation in healthy individuals.

The methods described herein may provide a significant increase in the sensitivity and specificity of detecting perturbations of methylation status based on a statistical analysis of the distribution of methylation status values determined for the individual subject and for the cohort, where the increased sensitivity and specificity of the statistical analyses arises from the large number of CpG loci evaluated and the prior expectation of correlated methylation states in the CpG loci within each “compact” genomic region. Even if the change in the average level of methylation in one or more “compact” genomic regions is small, sensitive statistical tests for the distribution of a methylation status metric (e.g., CpG loci methylation fraction values or DNA fragment-level methylation status values) can be used to discern the methylation signal.

In some instances, the disclosed methods and systems may comprise first defining a set of “compact” genomic regions that exhibit correlated methylation states in a specified cohort of health individuals, and then comparing the distribution of methylation status values determined for the same set of “compact” genomic regions in an individual subject to that for the cohort, where a difference between the distribution determined for the subject and the distribution determined for the cohort is indicative of a presence of the disease in the subject, or wherein a similarity between the distribution determined for the subject and the distribution determined for the cohort is indicative of an absence of the disease in the subject.

In some instances, the disclosed methods and systems may comprise first defining a set of “compact” genomic regions that exhibit correlated methylation states in a specified cohort of individuals having a specified disease, and then comparing the distribution of methylation status values determined for the same set of “compact” genomic regions in an individual subject to that for the cohort, where a difference between the distribution determined for the subject and the distribution determined for the cohort is indicative of an absence of the disease in the subject, or wherein a similarity between the distribution determined for the subject and the distribution determined for the cohort is indicative of a presence of the disease in the subject.

Disclosed herein are methods comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; determining, using the one or more processors, a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and performing, using the one or more processors, a statistical analysis to compare a distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the plurality of healthy individuals, wherein a difference between the distribution of methylation fraction values determined for the subject and the distribution of methylation fraction values determined for the plurality of healthy individuals is indicative of a presence of disease in the subject.

In some embodiments, the method further comprises comparing, using the one or more processors, the difference between the distribution of methylation fraction values determined for the subject and the distribution of methylation fraction values determined for the plurality of healthy individuals to a first predetermined threshold. In some embodiments, if the difference is greater than or equal to the first predetermined threshold, a disease-positive status is output by the one or more processors. If some embodiments, if the difference is less than the first predetermined threshold, a disease-negative status is output by the one or more processors.

In some embodiments, the statistical analysis comprises a t-test, a standardized mean difference test, a permutation test, a binned chi-squared test, or a Kolmogorov-Smirnov test. In some embodiments, the method further comprises use of a Kullbeck-Liebler divergence or informatics entropy-based approach to evaluate a difference between the distribution of methylation fraction values determined for the subject and the distribution of methylation fraction values determined for the plurality of healthy individuals.

In some embodiments, the subject is suspected of having or is determined to have cancer. In some embodiments, the cancer is comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the method further comprises treating the subject with an anti-cancer therapy. In some embodiments, the anti-cancer therapy comprises a targeted anti-cancer therapy. In some embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Sccmblix), atczolizumab (Tecentriq), avapritinib (Ayvakit), avclumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwle (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsctinib (Gavrcto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique.

In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer.

In some embodiments, one or more of the plurality of sequencing reads overlap one or more gene loci within one or more subgenomic intervals in the sample. In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1,CCND2, CCND3, CCNE1, CD22, CD274, CD70, CD74, CD79A, CD79B, CDC73, CDH1, CDK12, CDK4, CDK6, CDK8, CDKNIA, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2,TYRO3, U2AF1, VEGFA, VHL, WHSC1, WHSC1L1, WT1,XPO1, XRCC2, ZNF217, ZNF703, or any combination thereof.

In some embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-13, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, VEGFB, or any combination thereof.

In some embodiments, the method further comprises generating, by the one or more processors, a report indicating the detected difference between the distribution of methylation fraction values determined for the subject and the distribution of methylation fraction values determined for the plurality of healthy individuals, or a likelihood that the disease is present in the subject. In some embodiments, the method further comprises transmitting the report to a healthcare provider. In some embodiments, the report is transmitted via a computer network or a peer-to-peer connection.

Disclosed herein are methods for detecting disease in a subject, the method comprising: receiving, at one or more processors, sequence read data for a plurality of sequence reads derived from a sample from the subject; determining, using the one or more processors, a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and performing, using the one or more processors, a statistical analysis to compare a distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the plurality of healthy individuals, wherein a difference between the distribution of methylation fraction values determined for the subject and the distribution of methylation fraction values determined for the plurality of healthy individuals is indicative of a presence of disease in the subject.

In some embodiments, the method further comprises comparing, using the one or more processors, the difference between the distribution of methylation fraction values determined for the subject and the distribution of methylation fraction values determined for the plurality of healthy individuals to a first predetermined threshold. In some embodiments, if the difference is greater than or equal to the first predetermined threshold, a disease-positive status is output by the one or more processors. In some embodiments, if the difference is less than the first predetermined threshold, a disease-negative status is output by the one or more processors. In some embodiments, the difference between the distribution of methylation fraction values determined for the subject and the distribution of methylation fraction values determined for the plurality of healthy individuals comprises a difference in a mean, median, mode, 10th percentile, 25th percentile, 75th percentile, or 90th percentile of the methylation fraction values.

In some embodiments, the statistical analysis comprises a t-test, a standardized mean difference test, a permutation test, a binned chi-squared test, or a Kolmogorov-Smirnov test. In some embodiments, the method further comprises use of a Kullbeck-Liebler divergence or informatics entropy-based approach to evaluate a difference between the distribution of methylation fraction values determined for the subject and the distribution of methylation fraction values determined for the plurality of healthy individuals. In some embodiments, the plurality of CpG sites comprises all CpG sites within a given genomic region.

Also disclosed herein are methods for detecting disease in a subject, the method comprising: receiving, at one or more processors, sequence read data for a plurality of sequence reads derived from a sample from the subject; determining, using the one or more processors, a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a first plurality of individuals having a first disease; and performing, using the one or more processors, a statistical analysis to compare a distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the first plurality of individuals having the first disease, wherein a difference between the distribution of methylation fraction values determined for the subject and the distribution of methylation fraction values determined for the first plurality of individuals having the first disease is indicative of an absence of the first disease in the subject, or wherein a similarity between the distribution of methylation fraction values determined for the subject and the distribution of methylation fraction values determined for the first plurality of individuals having the first disease is indicative of a presence of the first disease in the subject.

In some embodiments, the method further comprises: determining, using the one or more processors, a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a second plurality of individuals having a second disease; and performing, using the one or more processors, a statistical analysis to compare the distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the second plurality of individuals having the second disease, wherein a difference between the distribution of methylation fraction values determined for the subject and the distribution of methylation fraction values determined for the second plurality of individuals having the second disease is indicative of an absence of the second disease in the subject, or wherein a similarity between the distribution of methylation fraction values determined for the subject and the distribution of methylation fraction values determined for the second plurality of individuals having the second disease is indicative of a presence of the second disease in the subject.

Disclosed herein are methods for detecting disease in a subject, the method comprising: receiving, at one or more processors, sequence read data for a plurality of sequence reads derived from a sample from the subject; determining, using the one or more processors, a fragment-level methylation status value for each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and performing, using the one or more processors, a statistical analysis to compare the fragment-level methylation status values determined for one or more genomic regions of the set of selected genomic regions in the subject to corresponding fragment-level methylation status values determined for one or more genomic regions of the set of selected genomic regions for the plurality of healthy individuals, wherein a difference between the fragment-level methylation fraction status values determined for one or more genomic regions for the subject and the fragment-level methylation fraction status values determined for the corresponding one or more genomic regions for the plurality of healthy individuals is indicative of a presence of disease in the subject.

In some embodiments, the method further comprises comparing, using the one or more processors, the difference between the fragment-level methylation status values determined for the one or more genomic regions for the subject and the fragment-level methylation fraction status values determined for the corresponding one or more genomic regions for the plurality of healthy individuals to a second predetermined threshold. In some embodiments, if the difference is greater than or equal to the second predetermined threshold, a disease-positive status is output by the one or more processors. In some embodiments, if the difference is less than the second predetermined threshold, a disease-negative status is output by the one or more processors. In some embodiments, the difference between the fragment-level methylation fraction status values determined for one or more genomic regions for the subject and the fragment-level methylation fraction status values determined for the corresponding one or more genomic regions for the plurality of healthy individuals comprises a difference in a mean, median, mode, 10th percentile, 25th percentile, 75th percentile, or 90th percentile of the fragment-level methylation status values.

In some embodiments, the statistical analysis comprises a t-test, a standardized mean difference test, a permutation test, a binned chi-squared test, or a Kolmogorov-Smirnov test.

Disclosed herein are methods for detecting disease in a subject, the method comprising: receiving, at one or more processors, sequence read data for a plurality of sequence reads derived from a sample from the subject; determining, using the one or more processors, a fragment-level methylation status value for each genomic region of a set of selected genomic regions based on the sequence read data, wherein each selected genomic region of the set comprises a plurality of CpG sites that are proximal to each other and that exhibit a consistent methylation status in sequence read data for a first plurality of individuals having a first disease; and performing, using the one or more processors, a statistical analysis to compare the fragment-level methylation status values determined for one or more genomic regions of the set of selected genomic regions in the subject to corresponding fragment-level methylation status values determined for one or more genomic regions of the set of selected genomic regions for the first plurality of individuals having the first disease, wherein a difference between the fragment-level methylation fraction status values determined for one or more genomic regions for the subject and the fragment-level methylation fraction status values determined for the corresponding one or more genomic regions for the first plurality of individuals having the first disease is indicative of an absence of the first disease in the subject, or wherein a similarity between the fragment-level methylation fraction status values determined for one or more genomic regions for the subject and the fragment-level methylation fraction status values determined for the corresponding one or more genomic regions for the first plurality of individuals having the first disease is indicative of a presence of the first disease in the subject.

In some embodiments, the method further comprises: determining, using the one or more processors, a fragment-level methylation status value for each genomic region of a set of selected genomic regions based on the sequence read data, wherein each selected genomic region of the set comprises a plurality of CpG sites that are proximal to each other and that exhibit a consistent methylation status in sequence read data for a second plurality of individuals having a second disease; and performing, using the one or more processors, a statistical analysis to compare the fragment-level methylation status values determined for the one or more genomic regions of the set of selected genomic regions in the subject to corresponding fragment-level methylation status values determined for one or more genomic regions of the set of selected genomic regions for a second plurality of individuals having a second disease, wherein a difference between the fragment-level methylation fraction status values determined for one or more genomic regions for the subject and the fragment-level methylation fraction status values determined for the corresponding one or more genomic regions for the second plurality of individuals having the second disease is indicative of an absence of the second disease in the subject, or wherein a similarity between the fragment-level methylation fraction status values determined for one or more genomic regions for the subject and the fragment-level methylation fraction status values determined for the corresponding one or more genomic regions for the second plurality of individuals having the second disease is indicative of a presence of the second disease in the subject.

In some embodiments of any of the methods disclosed herein, the set of selected genomic regions is selected based on the subject's age, sex, race, body mass index, smoking history, clinical history, family history, genetic predisposition for disease, diagnosed disease, polygenic risk score, or any combination thereof.

In some embodiments of nay of the methods disclosed herein, the set of selected genomic regions comprises at least 5, at least 10, at least 20, at least 30, at least 40, at least 50, at least 60, at least 70, at least 80, at least 90, at least 100, at least 200, at least 400, at least 600, at least 800, at least 1,000, at least 2,000, at least 4,000, at least 6,000, at least 8,000, at least 10,000, at least 20,000, at least 30,000, at least 40,000, at least 50,000, at least 60,000, at least 70,000, at least 80,000, at least 90,000, or at least 100,000 genomic regions.

In some embodiments of any of the methods disclosed herein, each selected genomic region comprises at least N CpG sites within a sequence of L bases in length. In some embodiments, N is 3, 4, or 5. In some embodiments, L is 50, 100, 150, 250, 300, or 350.

In some embodiments, of any of the methods disclosed herein, the methylation fraction values or fragment-level methylation fraction status values determined for the subject are used as input for a machine learning model configured to output a prediction of a probability that the subject has the disease. In some embodiments, the machine learning model comprises a supervised machine learning model. In some embodiments, the supervised machine learning model comprises a linear regression, random forest, support vector machine, artificial neural network or deep learning model. In some embodiments, the machine learning model is trained using a dataset comprising methylation fraction value data or fragment-level methylation fraction status value data for a cohort of subjects diagnosed with the disease and a cohort of healthy individuals.

In some embodiments, of any of the methods disclosed herein, the sample from the subject comprises a tissue biopsy sample, a liquid biopsy sample, a cervical swab sample, a pap smear samples, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the sample is a cervical swab or Pap smear sample and comprises cells from the subject's cervix.

In some embodiments of any of the methods disclosed herein, the methylation fraction value for each CpG site or the fragment-level methylation status value for each genomic region is determined based on sequence read data obtained using a nucleic acid sequencing method comprising the use of a bisulfite- or enzymatic-conversion reaction to convert non-methylated cytosine to uracil. In some embodiments, the methylation fraction value for each CpG site or the fragment-level methylation status value for each genomic region is determined based on sequence read data obtained using a nucleic acid sequencing method comprising the use of a bisulfite- or enzymatic-conversion reaction to convert methylated cytosine to uracil. In some embodiments, the methylation fraction value for each CpG site or the fragment-level methylation status value for each genomic region is determined based on sequence read data obtained using a targeted nucleic acid sequencing method that utilizes proteins comprising methyl-binding domains to capture genomic regions comprising methylated CpG sites.

In some embodiments of any of the methods disclosed herein, the disease, first disease, or second disease is cancer.

Also disclosed herein are methods for identifying informative genomic sub-regions, the method comprising: receiving, at one or more processors, sequence read data for a plurality of candidate genomic regions in samples from a plurality of healthy individuals; determining, using the one or more processors, a methylation fraction value for a plurality of CpG sites within each of the plurality of candidate genomic regions in each of the plurality of samples from the healthy individuals based on the sequence read data; determining, using the one or more processors, a fitted methylation fraction value for a plurality of CpG sites within each candidate genomic region by fitting genomic positions of the CpG sites and corresponding methylation fraction values of the plurality of CpG sites to a local regression model; identifying, using the one or more processors, one or more genomic sub-regions for each candidate genomic region by iteratively evaluating sequences of different length that: (i) exhibit a consistent methylation status in sequence read data for a plurality of healthy individuals, and (ii) comprise a plurality of CpG sites that are proximal to each other; identifying starting and ending genomic positions for each of the one or more genomic sub-regions; and assigning a label to each CpG site within the one or more genomic sub-regions based on their position within the genomic sub-region.

In some embodiments, identifying the one or more genomic sub-regions for each candidate genomic region comprises repeating, for each successive CpG site starting at a 5′-end of each candidate genomic region, the steps of: comparing a genomic position and a fitted methylation fraction value for a current CpG site to a genomic position and fitted methylation fraction value for a next CpG site; determining a separation distance between the current CpG site and the next CpG site; determining a methylation metric calculated based on the fitted methylation fraction values for a current genomic sub-region and in response to the separation distance being greater than a specified number of bases, d: (1) assigning the next CpG site to a new genomic sub-region, and (2) terminating a current genomic sub-region; or in response to the methylation metric being greater than a predetermined value, M, if the next CpG site were included in the current genomic sub-region: (1) assigning the next CpG site to a new genomic sub-region, and (2) terminating the current genomic sub-region; or assigning the next CpG site to the current genomic sub-region.

In some embodiments, the method further comprises assessing methylation fraction value data for the one or more identified genomic sub-regions for samples from a plurality of patients diagnosed with a specified disease to identify genomic sub-regions that may be used to differentiate between patients diagnosed with the disease and healthy individuals.

In some embodiments, the plurality of candidate genomic regions are selected from annotated genomic regions identified in a genomics database as exhibiting differential methylation status for a specified disease. In some embodiments, the plurality of candidate genomic regions are selected from genomic regions identified in a scientific publication as exhibiting differential methylation status for a specified disease. In some embodiments, the plurality of candidate genomic regions are selected from genomic regions identified in a genomics database that comprise cell type-specific markers, markers related to transcriptional programs, genes, or additional genomic features that have not been shown to exhibit differential methylation in a specified disease. In some embodiments, the additional genomic features comprise repeat elements, enhancers, promoters, DNase1 hypersensitive sites (DHSs), or any combination thereof. In some embodiments, the plurality of candidate genomic regions are selected from genomic regions associated with a functional pathway in a specified disease. In some embodiments, the specified disease is cancer and the genomic regions comprise tumor suppressor genes or oncogenes. In some embodiments, the specified disease is an immune system disorder and the genomic regions comprise major histocompatibility complex genes. In some embodiments, the specified disease comprises a cancer.

In some embodiments, the local regression model comprises a LOESS model. In some embodiments, the local regression model comprises a LOWESS model.

In some embodiments, the one or more genomic sub-regions identified comprise at least 5, at least 10, at least 20, at least 30, at least 40, at least 50, at least 60, at least 70, at least 80, at least 90, at least 100, at least 200, at least 400, at least 600, at least 800, at least 1,000, at least 2,000, at least 4,000, at least 6,000, at least 8,000, at least 10,000, at least 20,000, at least 30,000, at least 40,000, at least 50,000, at least 60,000, at least 70,000, at least 80,000, at least 90,000, or at least 100,000 genomic sub-regions.

In some embodiments, the specified number of bases, d, ranges from 10 bp to 1,000 bp. In some embodiments, the specified number of bases, d, ranges from 100 bp to 300 bp.

In some embodiments, the methylation metric is calculated based on the fitted methylation fraction values for all CpG sites in the current genomic sub-region and the fitted methylation fraction value for the next CpG site. In some embodiments, the methylation metric is calculated according to the relationship: methylation metric=max(mF)−min(mF) for all of the fitted methylation fraction (mF) values for CpG sites in the current sub-region.

In some embodiments, the value of M ranges from 0.05 to 0.3. In some embodiments, the value of M ranges from 0.1 to 0.15.

In some embodiments, each CpG located at the 5′-end of a candidate genomic region or at a 5′-end of a new genomic sub-region is assigned a label of “start”. In some embodiments, each CpG located at a 3′-end of a candidate genomic region or at a 3′-end of a new genomic sub-region is assigned a label of “end”. In some embodiments, each CpG located between a “start” CpG and an “end” CpG for a same genomic sub-region is assigned a label of “intermediate”.

In some embodiments, the identified genomic sub-regions are further extended by a short section of additional nucleic acid sequence at either end of the genomic sub-region defined by a corresponding pair of “start” and “end” CpGs.

In some embodiments, the samples comprise tissue biopsy samples, liquid biopsy samples, cervical swab samples, pap smear samples, normal controls, or any combination thereof. In some embodiments, the samples are liquid biopsy samples and comprise blood, plasma, cerebrospinal fluid, sputum, stool, urine, or saliva. In some embodiments, the samples are liquid biopsy samples and comprise circulating tumor cells (CTCs). In some embodiments, the samples are liquid biopsy samples and comprise cell-free DNA (cfDNA), circulating tumor DNA (ctDNA), or any combination thereof. In some embodiments, the samples are cervical swab or Pap smear samples and comprise cells from a cervix in a plurality of healthy individuals.

In some embodiments, the methylation fraction values are determined based on sequence read data obtained using a nucleic acid sequencing method comprising the use of a bisulfite- or enzymatic-conversion reaction to convert non-methylated cytosine to uracil. In some embodiments, the methylation fraction values are determined based on sequence read data obtained using a nucleic acid sequencing method comprising the use of a bisulfite- or enzymatic-conversion reaction to convert methylated cytosine to uracil. In some embodiments, the methylation fraction values are determined based on sequence read data obtained using a targeted nucleic acid sequencing method that utilizes proteins comprising methyl-binding domains to capture genomic regions comprising methylated CpG sites.

In some embodiments of any of the methods disclosed herein, the determination of a difference between the distribution of methylation fraction values determined for the subject and the distribution of methylation fraction values determined for a plurality of healthy individuals or diseased individuals is used to diagnose or confirm a diagnosis of disease in the subject. In some embodiments, the disease is cancer. In some embodiments, the cancer is acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck. a squamous non-small cell lung cancer, thyroid cancer, a thyroid carcinoma, urothelial cancer, a urothelial carcinoma, or Waldenstrom's macroglobulinemia.

In some embodiments, the method further comprising selecting an anti-cancer therapy to administer to the subject based on the determination of a difference between the distribution of methylation fraction values determined for the subject and the distribution of methylation fraction values determined for a plurality of healthy individuals or diseased individuals. In some embodiments, the method further comprises determining an effective amount of an anti-cancer therapy to administer to the subject based on the determination of a difference between the distribution of methylation fraction values determined for the subject and the distribution of methylation fraction values determined for a plurality of healthy individuals or diseased individuals. In some embodiments, the method further comprises administering the anti-cancer therapy to the subject based on the determination of a difference between the distribution of methylation fraction values determined for the subject and the distribution of methylation fraction values determined for a plurality of healthy individuals or diseased individuals. In some embodiments, the anti-cancer therapy comprises chemotherapy, radiation therapy, immunotherapy, a targeted therapy, or surgery.

Also disclosed herein are methods for diagnosing a disease, the method comprising: diagnosing that a subject has the disease based on a determination of a difference between the distribution of methylation fraction values determined for a subject and the distribution of methylation fraction values determined for a plurality of healthy individuals or diseased individuals for a sample from the subject, wherein the difference between the distribution of methylation fraction values determined for the subject and the distribution of methylation fraction values determined for a plurality of healthy individuals or diseased individuals is determined according to any of the methods described herein.

Disclosed herein are methods of selecting an anti-cancer therapy, the method comprising: responsive to determining a difference between the distribution of methylation fraction values determined for a sample from a subject and the distribution of methylation fraction values determined for samples from a plurality of healthy individuals or diseased individuals, selecting an anti-cancer therapy for the subject, wherein the difference between the distribution of methylation fraction values determined for the subject and the distribution of methylation fraction values determined for a plurality of healthy individuals or diseased individuals for a sample from the subject is determined according to any of the methods described herein.

Disclosed herein are methods of treating a cancer in a subject, comprising: responsive to determining a difference between the distribution of methylation fraction values determined for a sample from a subject and the distribution of methylation fraction values determined for samples from a plurality of healthy individuals or diseased individuals, administering an effective amount of an anti-cancer therapy to the subject, wherein the difference between the distribution of methylation fraction values determined for the sample from the subject and the distribution of methylation fraction values determined for samples from a plurality of healthy individuals or diseased individuals is determined according to any of the methods described herein.

Disclosed herein are methods for monitoring cancer progression or recurrence in a subject, the method comprising: determining a first difference between the distribution of methylation fraction values determined for a sample from a subject and the distribution of methylation fraction values in samples from a plurality of healthy individuals or diseased individuals at a first time point according to any of the methods described herein; determining a second difference between the distribution of methylation fraction values determined for a sample from the subject and the distribution of methylation fraction values for the plurality of healthy individuals or diseased individuals at a second time point; and comparing the first determination to the second determination, thereby monitoring the cancer progression or recurrence. In some embodiments, the second determination for the second sample is determined according to any of the methods described herein. In some embodiments, the method further comprises selecting an anti-cancer therapy for the subject in response to the cancer progression. In some embodiments, the method further comprises administering an anti-cancer therapy to the subject in response to the cancer progression. In some embodiments, the method further comprises adjusting an anti-cancer therapy for the subject in response to the cancer progression. In some embodiments, the method further comprises adjusting a dosage of the anti-cancer therapy or selecting a different anti-cancer therapy in response to the cancer progression. In some embodiments, the method further comprises administering the adjusted anti-cancer therapy to the subject. In some embodiments, the first time point is before the subject has been administered an anti-cancer therapy, and wherein the second time point is after the subject has been administered the anti-cancer therapy.

In some embodiments, the subject has a cancer, is at risk of having a cancer, is being routine tested for cancer, or is suspected of having a cancer. In some embodiments, the cancer is a solid tumor. In some embodiments, the cancer is a hematological cancer. In some embodiments, the anti-cancer therapy comprises chemotherapy, radiation therapy, immunotherapy, a targeted therapy, or surgery.

In some embodiments of any of the methods disclosed herein, the method further comprises determining, identifying, or applying a value for the difference between the distribution of methylation fraction values determined for a sample from a subject and the distribution of methylation fraction values in samples from a plurality of healthy individuals or diseased individuals as a diagnostic value associated with the sample from the subject.

In some embodiments of any of the methods disclosed herein, the method further comprises generating a genomic profile for the subject based on the determination of the difference between the distribution of methylation fraction values determined for a sample from a subject and the distribution of methylation fraction values in samples from a plurality of healthy individuals or diseased individuals. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test. In some embodiments, the method further comprises selecting an anti-cancer therapy, administering an anti-cancer therapy, or applying an anti-cancer therapy to the subject based on the generated genomic profile.

The method of any one of claims 34 to 69, wherein the determination of the difference between the distribution of methylation fraction values determined for a sample from a subject and the distribution of methylation fraction values in samples from a plurality of healthy individuals or diseased individuals is used in making suggested treatment decisions for the subject.

The method of any one of claims 34 to 69, wherein the determination of the difference between the distribution of methylation fraction values determined for a sample from a subject and the distribution of methylation fraction values in samples from a plurality of healthy individuals or diseased individuals is used in applying or administering a treatment to the subject.

Disclosed herein are systems comprising: one or more processors; and a memory communicatively coupled to the one or more processors and configured to store instructions that, when executed by the one or more processors, cause the system to perform any of the methods described herein.

Also disclosed herein are non-transitory computer-readable storage media storing one or more programs, the one or more programs comprising instructions, which when executed by one or more processors of a system, cause the system to perform any of the methods described herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for identifying informative genomic sub-regions, according to one embodiment described herein.

FIG. 2 provides a non-limiting example of a process flowchart for determining a likelihood that a subject has a disease, according to one embodiment described herein.

FIG. 3 provides a non-limiting example of a process flowchart for determining a likelihood that a subject has a disease, according to another embodiment described herein.

FIG. 4 provides a non-limiting example of a process flowchart for determining a likelihood that a subject has a disease, according to another embodiment described herein.

FIG. 5 provides a non-limiting example of a process flowchart for determining a likelihood that a subject has a disease, according to another embodiment described herein.

FIG. 6 depicts an exemplary computing device or system in accordance with one embodiment of the present disclosure.

FIG. 7 depicts an exemplary computer system or computer network, in accordance with some instances of the systems described herein.

FIG. 8 provides a non-limiting example of a plot of CpG loci methylation fraction values (raw and fitted) as a function of genomic position in lung tissue DNA.

FIG. 9 provides a non-limiting example of a plot of CpG loci methylation fraction values as a function of genomic position in lung tissue DNA.

FIG. 10 provides a non-limiting schematic illustration of the calculation of CpG loci methylation fraction values or DNA fragment-level methylation status values for genomic regions or sub-regions based on sequence read data.

FIG. 11 provides a non-limiting example of a plot of fitted CpG loci methylation fraction values as a function of genomic position that illustrates the subdivision of a candidate genomic region from chromosome 12 to identify a “compact” genomic region.

FIG. 12 provides a non-limiting example of a plot of fitted CpG loci methylation fraction values as a function of genomic position that illustrates the subdivision of a candidate genomic region from chromosome 8 to identify a “compact” genomic region.

### DETAILED DESCRIPTION

Methods and systems for the detection of disease, e.g., cancer, based on DNA methylation status are described that comprise first defining a set of “compact” genomic regions that exhibit correlated methylation states in a specified cohort of individuals, e.g., healthy individuals, and then comparing the distribution of methylation status values determined for the same set of “compact” genomic regions in an individual subject to that for the cohort to detect perturbations in methylation status and determine the likelihood that a disease, e.g., cancer, is present in the subject. As described herein, a “compact” genomic region is a segment of the genome that includes a plurality of CpG sites in relatively close proximity and that exhibits relatively consistent and correlated levels of CpG methylation in healthy individuals.

The methods described herein may provide a significant increase in the sensitivity and specificity of detecting perturbations of methylation status based on a statistical analysis of the distribution of methylation status values determined for the individual subject and for the cohort, where the increased sensitivity and specificity of the statistical analyses arises from the large number of CpG loci evaluated and the prior expectation of correlated methylation states in the CpG loci within each “compact” genomic region. Even if the change in the average level of methylation in one or more “compact” genomic regions is small, sensitive statistical tests for the distribution of a methylation status metric (e.g., CpG loci methylation fraction values or DNA fragment-level methylation status values) can be used to discern the methylation signal.

In some instances, the disclosed methods and systems may comprise first defining a set of “compact” genomic regions that exhibit correlated methylation states in a specified cohort of individuals having a specified disease, and then comparing the distribution of methylation status values determined for the same set of “compact” genomic regions in an individual subject to that for the cohort, where a difference between the distribution determined for the subject and the distribution determined for the cohort is indicative of an absence of the disease in the subject, or wherein a similarity between the distribution determined for the subject and the distribution determined for the cohort is indicative of a presence of the disease in the subject.

In some instances, for example, methods are described that comprise receiving sequence read data for a plurality of sequence reads derived from a sample from the subject; determining a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and performing a statistical analysis to compare a distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the plurality of healthy individuals, wherein a difference between the distribution of methylation fraction values determined for the subject and the distribution of methylation fraction values determined for the plurality of healthy individuals is indicative of a presence of disease in the subject.

In some instances, as another example, methods are described that comprise receiving sequence read data for a plurality of sequence reads derived from a sample from the subject; determining a fragment-level methylation status value for each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and performing a statistical analysis to compare the fragment-level methylation status values determined for one or more genomic regions of the set of selected genomic regions in the subject to corresponding fragment-level methylation status values determined for one or more genomic regions of the set of selected genomic regions for the plurality of healthy individuals, wherein a difference between the fragment-level methylation fraction status values determined for one or more genomic regions for the subject and the fragment-level methylation fraction status values determined for the corresponding one or more genomic regions for the plurality of healthy individuals is indicative of a presence of disease in the subject.

In some instances, the method may further comprise comparing the difference between the distribution of methylation fraction values determined for the subject and the distribution of methylation fraction values determined for the plurality of healthy individuals to a predetermined threshold. In some instances, if the difference is greater than or equal to the predetermined threshold, a disease-positive status is output. In some instances, if the difference is less than the predetermined threshold, a disease-negative status is output. In some instances, the method may further comprise comparing the difference between the distribution of methylation fraction values determined for the subject and the distribution of methylation fraction values determined for the plurality of healthy individuals to two or more predetermined thresholds, e.g., to distinguish between a disease-positive, disease-negative, and disease-ambiguous state.

As noted above, the disclosed methods and systems may provide a significant increase in the sensitivity and specificity of detecting perturbations of methylation status in genomic data due to the large number of CpG loci that may be evaluated and the prior expectation of correlated methylation states in the CpG loci within each “compact” genomic region selected for evaluation.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About”′ and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Identifying Informative Genomic Regions

The disclosed methods and systems enable significant increases in the sensitivity and specificity of detecting perturbations of methylation status in genomic data due to the large number of CpG loci that may be evaluated and the prior expectation of correlated methylation states in the CpG loci within each “compact” genomic region selected for evaluation, where a “compact” (or “informative”) genomic region is a segment of the genome that includes a plurality of CpG sites in relatively close proximity and that exhibits relatively consistent and correlated levels of CpG methylation in healthy individuals.

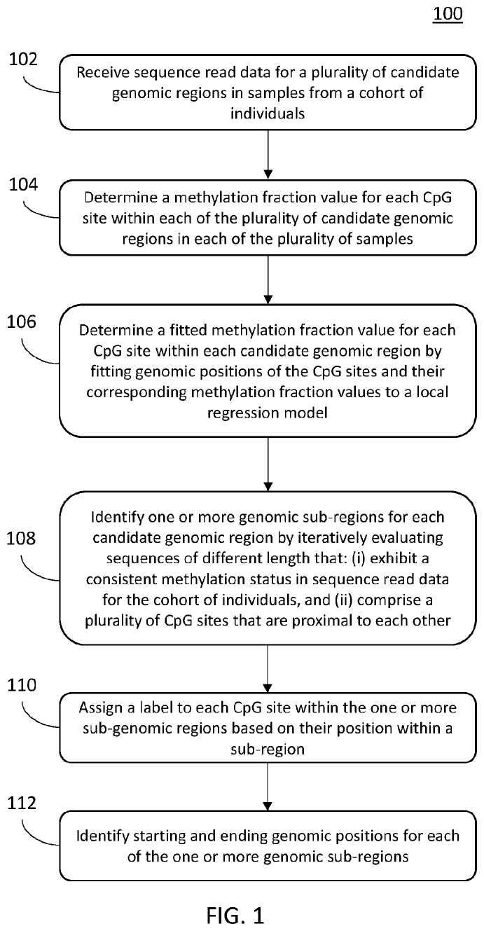

FIG. 1 provides a non-limiting example of a flowchart for a process 100 for identifying informative genomic sub-regions. Process 100 (or any of the other processes described herein) can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, sequence read data is received for a plurality of candidate genomic regions in samples from a cohort of individuals, e.g., a cohort of health individuals. In some instances, the samples may comprise tissue biopsy samples, liquid biopsy samples, cervical swab samples, pap smear samples, normal controls, or any combination thereof.

In some instances, the samples may be liquid biopsy samples, and may comprise blood, plasma, cerebrospinal fluid, sputum, stool, urine, or saliva. In some instances, the samples may be liquid biopsy samples, and may comprise circulating tumor cells (CTCs). In some instances, the samples may be liquid biopsy samples, and may comprise cell-free DNA (cfDNA), circulating tumor DNA (ctDNA), or any combination thereof.

In some instances, the samples may be cervical swab or Pap smear samples, and may comprise cells from a cervix in a plurality of healthy individuals.

In some instances, the sequence read data may be obtained using a nucleic acid sequencing method comprising the use of a bisulfite- or enzymatic-conversion reaction to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions may be utilized to convert non-methylated cytosine to uracil (or to convert methylated cytosine to uracil), for example, enzymatic deamination of non-methylated cytosine using APOBEC to form uracil (see, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

In some instances, the sequence read data may be obtained using a targeted nucleic acid sequencing method that utilizes proteins comprising methyl-binding domains to capture genomic regions comprising methylated CpG sites.

In some instances, the plurality of candidate genomic regions may be selected, for example, from annotated genomic regions identified in a genomics database as exhibiting differential methylation status for a specified disease. In some instances, the plurality of candidate genomic regions may be selected from annotated genomic regions identified in genomics databases that comprise cell type specific markers, markers related to transcriptional programs, genes and other genomic features (repeat elements, enhancers, promoters, DNase 1 hypersensitive sites (DHSs), etc.) that have not necessarily been shown previously to exhibit differential methylation in a specific disease. In some instances, the plurality of candidate genomic regions may be selected from genomic regions identified in a scientific publication as exhibiting differential methylation status for a specified disease, e.g., a cancer.

In some instances, the plurality of candidate genomic regions may be selected from genomic regions associated with a functional pathway in a specified disease. In some instances, the specified disease may be cancer, and the genomic regions may comprise, for example, tumor suppressor genes or oncogenes. In some instances, the specified disease may be an immune system disorder, and the genomic regions may comprise major histocompatibility complex genes.

In some instances, the cohort of individuals may comprise a cohort of diseased individuals, e.g., individuals diagnoses with a specific disease. In some instances, the specific disease may be a cancer or other genetic disease.

At step 104 in FIG. 1, a methylation fraction value is determined for each CpG site within each of the plurality of candidate genomic regions in each of the plurality of samples. In some instances, the methylation fraction value for each CpG site (i.e., a locus-specific methylation fraction value) may be calculated as the fraction of sequence reads covering the given CpG site for which the CpG site is methylated.

At step 106 in FIG. 1, a fitted methylation fraction value is determined for each CpG site within each candidate genomic region by fitting the genomic positions of the CpG sites and their corresponding methylation fraction values (determined in the previous step) to a local regression model. In some instances, the fitting step may provide for a more accurate estimate of the methylation fraction value for a given CpG site by taking into account the methylation fraction data for the given CpG site in a given candidate genomic region across all samples in the cohort.

In some instances, rather than fitting the genomic positions of the CpG sites and their corresponding methylation fraction values to a local regression model, one may calculate, e.g., a mean, median, or mode for the methylation fraction value for a given CpG site.

Local regression is a generalization of a combined moving average calculation and polynomial regression used to fit localized subsets of data to simple functions to generate a global function that describes the deterministic component of the variation in the data on a point-by-point basis. In some instances, for example, the local regression model may comprise a Locally Estimated Scatterplot Smoothing (LOESS) model. In some instances, the local regression model comprises a Locally Weighted Scatterplot Smoothing (LOWESS) model.

At step 108 in FIG. 1, one or more genomic sub-regions are identified for each candidate genomic region by iteratively evaluating sequences of different length that: (i) exhibit a consistent methylation status in sequence read data for the cohort of individuals, and (ii) comprise a plurality of CpG sites that are proximal to each other.

In some instances, for example, the step of identifying the one or more genomic sub-regions for each candidate genomic region may comprise repeating, for each successive CpG site starting at a 5′-end of each candidate genomic region, the steps of: (i) comparing a genomic position and a fitted methylation fraction value for a current CpG site to a genomic position and fitted methylation fraction value for a next CpG site; (ii) determining a separation distance between the current CpG site and the next CpG site; (iii) determining a methylation metric calculated based on the fitted methylation fraction values for a current genomic sub-region and (iv) in response to the separation distance being greater than a specified number of bases, d: (1) assigning the next CpG site to a new genomic sub-region, and (2) terminating a current genomic sub-region; or (v) in response to the methylation metric being greater than a predetermined value, M, if the next CpG site were included in the current genomic sub-region: (1) assigning the next CpG site to a new genomic sub-region, and (2) terminating the current genomic sub-region; or (vi) assigning the next CpG site to the current genomic sub-region.

In some instances, the specified number of bases, d, may range from 10 bp to 1,000 bp. In some instances, the specified number of bases, d, may be at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1,000 bp. In some instances, the specified number of bases, d, may be at most 1,000, at most 900, at most 800, at most 700, at most 600, at most 500, at most 400, at most 300, at most 200, at most 100, at most 90, at most 80, at most 70, at most 60, at most 50, at most 40, at most 30, at most 20, or at most 10 bp. Any of the lower and upper values described in this paragraph may be combined to form a range included within the present disclosure, for example, in some instances the specified number of bases, d, may range from 100 bp to 300 bp. Those of skill in the art will recognize that the specified number of bases, d, may have any value within this range, e.g., 112 bp.

In some instances, the methylation metric may be calculated based on the fitted methylation fraction values for all CpG sites in the current genomic sub-region and the fitted methylation fraction value for the next CpG site. In some instances, the methylation metric may be calculated according to the relationship:

methylation metric=max(mF)−min(mF)

for all of the fitted methylation fraction (mF) values for CpG sites in the current sub-region.

In some instances, the value of M may range from 0.05 to 0.3. In some instances, the value of M may be at least 0.05, at least 0.1, at least 0.15, at least 0.2, at least 0.25, or at least 0.3. In some instances, the value of M may be at most 0.3, at most 0.25, at most 0.2, at most 0.15, at most 0.1, or at most 0.05. Any of the lower and upper values described in this paragraph may be combined to form a range included within the present disclosure, for example, in some instances the value of M may range from 0.1 to 0.15. Those of skill in the art will recognize that the value of M may have any value within this range, e.g., 0.23.

At step 110 in FIG. 1, a label is assigned to each CpG site within the one or more subgenomic regions based on their position within the genomic sub-region. For example, in some instances, each CpG located at the 5′-end of a candidate genomic region or at a 5′-end of a new genomic sub-region is assigned a label of “start”. In some instances, each CpG located at a 3′-end of a candidate genomic region or at a 3′-end of a new genomic sub-region is assigned a label of “end”. In some instances, each CpG located between a “start” CpG and an “end” CpG for a same genomic sub-region is assigned a label of “intermediate”.

At step 112 in FIG. 1, starting and ending genomic positions are identified for each of the one or more genomic sub-regions.

In some instances, the identified genomic sub-regions are optionally extended by a short section of additional nucleic acid sequence at either end of the genomic sub-region defined by a corresponding pair of “start” and “end” CpGs in order to ensure that both genomic positions in a given CpG dinucleotide (i.e., the C and the G nucleotides) are included in the sub-region interval regardless of the downstream sequence analysis method used. For example, in some instances, the short section of additional nucleic acid sequence included at either end of the genomic sub-region may comprise 1, 2, 3, 4, 5,6, 7, 8, 9, 10, or more than 10 bp at either or both ends of the genomic sub-region as originally identified.

In some instances, the disclosed methods for identifying informative (or “compact”) genomic sub-regions may further comprising assessing methylation fraction value data for the one or more identified genomic sub-regions for samples from a plurality of patients diagnosed with a specified disease, e.g., cancer or another genetic disease, to identify genomic sub-regions that may be used to differentiate between patients diagnosed with the disease and healthy individuals.

In some instances, the disclosed methods for identifying informative (or “compact”) genomic sub-regions may comprise identifying one or more genomic sub-regions for one or more of the original candidate genomic regions. In some instances, the one or more genomic sub-regions identified comprise at least 5, at least 10, at least 20, at least 30, at least 40, at least 50, at least 60, at least 70, at least 80, at least 90, at least 100, at least 200, at least 400, at least 600, at least 800, at least 1,000, at least 2,000, at least 4,000, at least 6,000, at least 8,000, at least 10,000, at least 20,000, at least 30,000, at least 40,000, at least 50,000, at least 60,000, at least 70,000, at least 80,000, at least 90,000, or at least 100,000 genomic sub-regions.

In some instances, the informative (or “compact”) genomic sub-regions, once identified, may be referred to as informative (or “compact”) genomic regions for use in the disease detection methods described herein. The focus on selected sets of “compact” genomic regions for evaluation of DNA methylation data allows one to reduce noise in baseline methylation status (due to the focus on selected regions of the genome that are consistently methylated) and to improve signal (as a result of examining a larger number of CpG loci), which leads to better statistics when comparing, e.g., DNA methylation data for an individual subject (e.g., a patient) to that for a cohort of healthy and/or diseased individuals (i.e., individuals diagnosed with a specified disease, such as a specified cancer).

### Methods for Detecting Disease Based on DNA Methylation Data

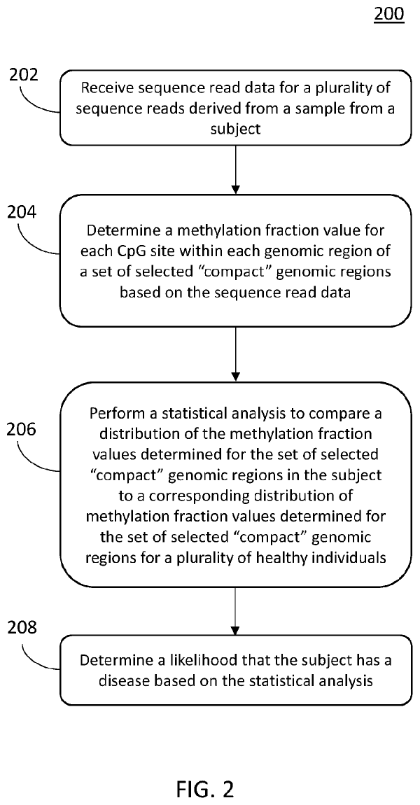

FIG. 2 provides a non-limiting example of a flowchart for a process 200 for detecting a disease or determining a likelihood that a subject has a disease. Process 200 (or any of the other processes described herein) can be performed, for example, using one or more electronic devices implementing a software platform. In some examples, process 200 is performed using a client-server system, and the blocks of process 200 are divided up in any manner between the server and a client device. In other examples, the blocks of process 200 are divided up between the server and multiple client devices. Thus, while portions of process 200 are described herein as being performed by particular devices of a client-server system, it will be appreciated that process 200 is not so limited. In other examples, process 200 is performed using only a client device or only multiple client devices. In process 200, some blocks are, optionally, combined, the order of some blocks is, optionally, changed, and some blocks are, optionally, omitted. In some examples, additional steps may be performed in combination with the process 200. Accordingly, the operations as illustrated (and described in greater detail below) are exemplary by nature and, as such, should not be viewed as limiting.

At step 202 in FIG. 2, sequence read data is received for a plurality of sequence reads derived from a sample from a subject.

At step 204 in FIG. 2, a methylation fraction value may be determined for each CpG site within each genomic region of a set of selected “compact” genomic regions (e.g., genomic regions that include a plurality of CpG sites that are proximal to each other and that exhibit a consistent level of CpG methylation in a cohort of individuals, e.g., a cohort of healthy individuals) based on the sequence read data. In some instances, the methylation fraction value for each CpG site (i.e., a locus-specific methylation fraction value) may be calculated as the fraction of sequence reads covering the given CpG site for which the CpG site is methylated.

At step 206 in FIG. 2, a statistical analysis may be performed to compare a distribution of the methylation fraction values determined for each CpG site within one of more genomic regions of the set of selected “compact” genomic regions in the subject to a corresponding distribution of methylation fraction values determined for each CpG site within one or more genomic regions of the set of selected “compact” genomic regions for the cohort of individuals, e.g., health individuals. In some instances, the statistical analysis may comprise a t-test, a standardized mean difference test, a permutation test, a binned chi-squared test, or a Kolmogorov-Smirnov test.

At step 208 in FIG. 2, a likelihood that the subject has a disease is determined based on the statistical analysis. For example, in some instances, a difference between the distribution of methylation fraction values determined for the subject and the distribution of methylation fraction values determined for the cohort of healthy individuals may be indicative of a presence of disease in the subject.

In some instances, the method may further comprise comparing the difference between the distribution of methylation fraction values determined for the subject and the distribution of methylation fraction values determined for the cohort of healthy individuals to a predetermined threshold. In some instances, if the difference is greater than or equal to the predetermined threshold, a disease-positive status may be output. In some instances, if the difference is less than the predetermined threshold, a disease-negative status may be output. In some instances, the method may further comprise comparing the difference between the distribution of methylation fraction values determined for the subject and the distribution of methylation fraction values determined for the cohort of healthy individuals to two or more predetermined thresholds, e.g., to distinguish between a disease-positive, disease-negative, and disease-ambiguous state.

In some instances, cut-off thresholds may be determined, e.g., by varying a candidate threshold over the range of differences in methylation fraction value distributions and computing false-positive-rate (FPR) and true-positive-rate (TPR) for each threshold value, which allows the construction of a receiver operating characteristic (ROC) curve. The “optimal” point on the ROC curve can be chosen in different ways, e.g., according to a pre-specified FPR or TPR. Other approaches can include determining a maximum value for metrics such as (TPR−FPR) or sqrt(TPR*FPR).

In some instances, the difference between the distribution of methylation fraction values determined for the subject and the distribution of methylation fraction values determined for the cohort of healthy individuals may comprise a difference in a mean, median, mode, 10th percentile, 25th percentile, 75th percentile, or 90th percentile of the methylation fraction values.

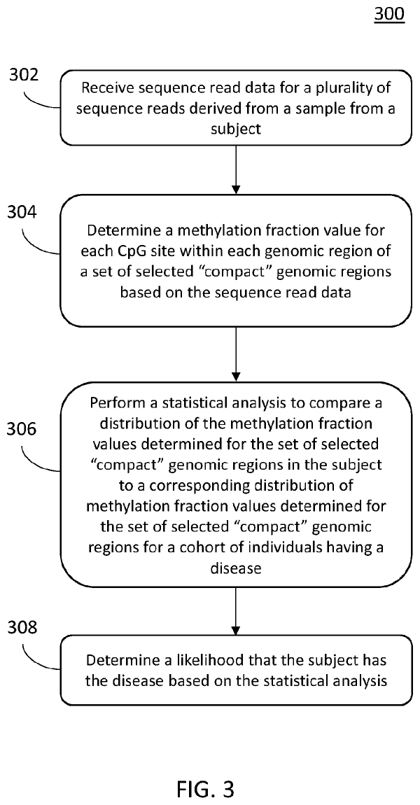

FIG. 3 provides a non-limiting example of a flowchart for another process 300 for detecting a disease or determining a likelihood that a subject has a disease.

At step 302 in FIG. 3, sequence read data is received for a plurality of sequence reads derived from a sample from a subject.

At step 304 in FIG. 3, a methylation fraction value is determined for each CpG site within each genomic region of a set of selected “compact” genomic regions (e.g., genomic regions that include a plurality of CpG sites that are proximal to each other and that exhibit a consistent level of CpG methylation in a cohort of individuals, e.g., a cohort of diseased individuals, e.g., individuals diagnosed with a specified disease such as a cancer) based on the sequence read data. In some instances, the methylation fraction value for each CpG site (i.e., a locus-specific methylation fraction value) may be calculated as the fraction of sequence reads covering the given CpG site for which the CpG site is methylated. In some instances, the set of selected “compact” genomic regions to be evaluated may be different for different diseases.

At step 306 in FIG. 3, a statistical analysis is performed to compare a distribution of the methylation fraction values determined for each CpG site within one or more genomic regions of the set of selected “compact” genomic regions in the subject to a corresponding distribution of methylation fraction values determined for each CpG site within one or more genomic regions of the set of selected “compact” genomic regions for the cohort of individuals having the specified disease. In some instances, the statistical analysis may comprise a t-test, a standardized mean difference test, a permutation test, a binned chi-squared test, or a Kolmogorov-Smirnov test.

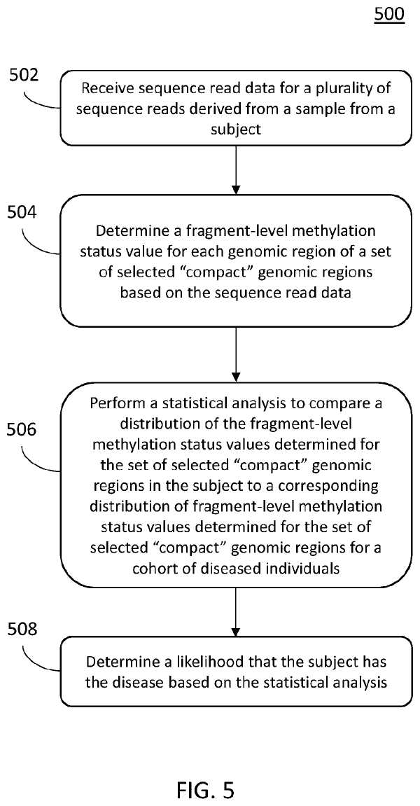

In some instances, the set of selected “compact” genomic regions to be evaluated may be the same as that identified for a cohort of healthy patients, and the statistical analysis of the methylation fraction values for a sample from an individual may be performed to compare their distribution to both a distribution of the methylation fraction values determined for the healthy cohort and to a distribution of the methylation fraction values determined for one or more disease cohort(s) to determine if the sample is closer to the healthy case, a disease case, or is of ambiguous status based on a set of methylation fraction value metrics. A similar approach may be take with the fragment-level methylation status value-based methods described below with respect to FIG. 5.

At step 308 in FIG. 3, a likelihood that the subject has a disease is determined based on the statistical analysis. For example, in some instances, a difference between the distribution of methylation fraction values determined for the subject and the distribution of methylation fraction values determined for the cohort of individuals having the disease is indicative of an absence of the first disease in the subject. In some instances, a similarity between the distribution of methylation fraction values determined for the subject and the distribution of methylation fraction values determined for the cohort of individuals having the disease is indicative of a presence of the disease in the subject.

In some instances, the method may further comprise comparing the difference between the distribution of methylation fraction values determined for the subject and the distribution of methylation fraction values determined for the cohort of diseased individuals to a predetermined threshold. In some instances, if the difference is greater than or equal to the predetermined threshold, a disease-negative status may be output. In some instances, if the difference is less than the predetermined threshold, a disease-positive status may be output. In some instances, the method may further comprise comparing the difference between the distribution of methylation fraction values determined for the subject and the distribution of methylation fraction values determined for the cohort of diseased individuals to two or more predetermined thresholds, e.g., to distinguish between a disease-positive, disease-negative, and disease-ambiguous state.

In some instances, the difference between the distribution of methylation fraction values determined for the subject and the distribution of methylation fraction values determined for the cohort of diseased individuals may comprise a difference in a mean, median, mode, 10th percentile, 25th percentile, 75th percentile, or 90th percentile of the methylation fraction values.

In some instances, the method may further comprise: determining a methylation fraction value for each CpG site within each genomic region of a set of selected “compact” genomic regions based on the sequence read data, wherein each selected “compact” genomic region of the set comprises a plurality of CpG sites that are proximal to each other and that exhibit a consistent methylation status in sequence read data for a second cohort of diseased individuals, e.g., individuals diagnosed with a second specified disease such as a second cancer; and performing a statistical analysis (e.g., the same statistical analysis used with the first cohort of diseased individuals or a different statistical analysis) to compare the distribution of the methylation fraction values determined for each CpG site within one or more genomic regions of the set of selected “compact” genomic regions in the subject to a corresponding distribution of methylation fraction values determined for each CpG site within one or more genomic regions of the set of selected “compact” genomic regions for the second cohort of diseased individuals, where a difference between the distribution of methylation fraction values determined for the subject and the distribution of methylation fraction values determined for the second cohort of diseased individuals is indicative of an absence of the second disease in the subject, or where a similarity between the distribution of methylation fraction values determined for the subject and the distribution of methylation fraction values determined for the second cohort of diseased individuals is indicative of a presence of the second disease in the subject.

In some instances, the method may be repeated for a third, fourth, fifth disease, etc., using data for a third, fourth, fifth, etc., cohort of diseased individuals.

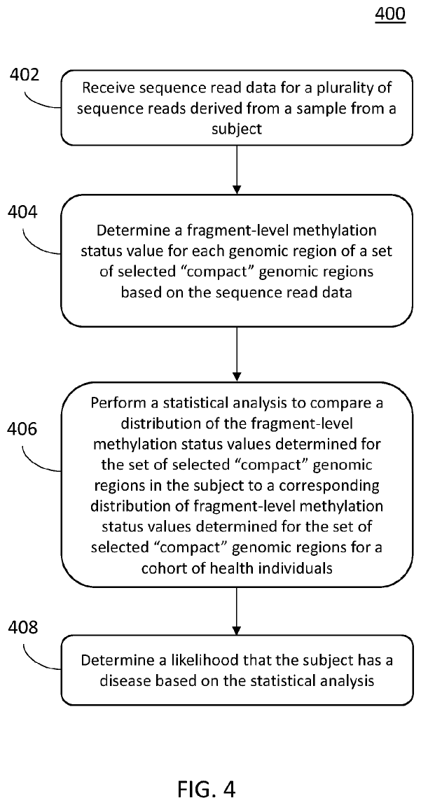

FIG. 4 provides a non-limiting example of a flowchart for a process 400 for detecting a disease or determining a likelihood that a subject has a disease.

At step 402 in FIG. 4, sequence read data is received for a plurality of sequence reads derived from a sample from a subject.

At step 404 in FIG. 4, a fragment-level methylation status value is determined for each genomic region of a set of selected “compact” genomic regions (e.g., genomic regions that include a plurality of CpG sites that are proximal to each other and that exhibit a consistent level of CpG methylation in a cohort of individuals, e.g., a cohort of health individuals) based on the sequence read data, where a “fragment” comprises a complementary pair of forward and reverse sequence reads.

In some instances, the fragment-level methylation status value may be calculated as the fraction of the total number of CpG sites within each genomic region that are methylated. In some instances, the fragment-level methylation status value may be calculated based on individual sequence read pairs (e.g., complementary pairs of forward and reverse sequence reads) that overlap with all or a portion of a specified genomic region. In some instances, the fragment-level methylation status value may be calculated based on the set of paired sequence reads that collectively overlap a specified genomic region.

At step 406 in FIG. 4, a statistical analysis is performed to compare a distribution of the fragment-level methylation status values determined for one or more genomic regions of the set of selected “compact” genomic regions in the subject to a corresponding distribution of fragment-level methylation status values determined for one or more genomic regions of the set of selected “compact” genomic regions for the cohort of health individuals. In some instances, the statistical analysis may comprise a t-test, a standardized mean difference test, a permutation test, a binned chi-squared test, or a Kolmogorov-Smirnov test.

Alternatively, in some instances a methylation fraction value may be determined for each CpG locus in each fragment (e.g., pair of forward and reverse sequence reads) at step 404, and a statistical analysis (e.g., a Binomial test) may be performed at step 406 to compare the methylation status of a fragment under test (FUT) for the subject to the methylation status of fragments from healthy individuals in the same compact genomic region. For example, assume that each fragment comprises N CpG loci and that m of them are methylated. One can collect all of the fragments for a specified compact genomic region in samples from a cohort of healthy individuals, sum up all values of N and m, and compute a probability that a given locus is methylated in the compact genomic region in healthy subjects as P=Σᵢ(mᵢ)/Σᵢ(Nᵢ). In some instances, for example, one can then can take a given fragment for a sample from a subject of unknown disease status and run a Binomial test based on (m, N, P) to determine how likely is it that the given fragment came from the distribution of healthy subject fragments. In some instances, one can use Kullbeck-Liebler divergence to perform this fragment assessment as m and N are discrete values.

In some instances, one may compute fragment-level methylation status values and compare their distributions for an unknown sample and a “Panel of Normals” (PoN) sample using, e.g., a Kolmogorov-Smirnov test.

In some instances, the method may further comprise use of a Kullbeck-Liebler divergence or informatics entropy-based approach to evaluate the difference between the distribution of fragment-level methylation status values determined for the subject and the distribution of fragment-level methylation status values determined for the cohort of health individuals.

At step 408 in FIG. 4, a likelihood that the subject has a disease is determined based on the statistical analysis. For example, in some instances, a difference between the distribution of fragment-level methylation status values determined for the subject and the distribution of fragment-level methylation status values determined for the cohort of healthy individuals may be indicative of a presence of disease in the subject.

In some instances, the method may further comprise comparing the difference between the distribution of fragment-level methylation status values determined for the subject and the distribution of fragment-level methylation status values determined for the cohort of healthy individuals to a predetermined threshold. In some instances, if the difference is greater than or equal to the predetermined threshold, a disease-positive status may be output. In some instances, if the difference is less than the predetermined threshold, a disease-negative status may be output. In some instances, the method may further comprise comparing the difference between the distribution of fragment-level methylation status values determined for the subject and the distribution of fragment-level methylation status values determined for the cohort of healthy individuals to two or more predetermined thresholds, e.g., to distinguish between a disease-positive, disease-negative, and disease-ambiguous state.

In some instances, the difference between the distribution of fragment-level methylation status values determined for the subject and the distribution of fragment-level methylation status values determined for the cohort of healthy individuals may comprise a difference in a mean, median, mode, 10th percentile, 25th percentile, 75th percentile, or 90th percentile of the fragment-level methylation status values.

FIG. 5 provides a non-limiting example of a flowchart for a process 500 for detecting a disease or for determining a likelihood that a subject has a disease.

At step 502 in FIG. 5, sequence read data is received for a plurality of sequence reads derived from a sample from a subject.

At step 504 in FIG. 5, a fragment-level methylation status value is determined for each genomic region of a set of selected “compact” genomic regions based on the sequence read data, where each selected “compact” genomic region of the set comprises a plurality of CpG sites that are proximal to each other and that exhibit a consistent methylation status in sequence read data for a cohort of individuals having a disease (e.g., individuals diagnosed with a specified disease such as cancer), and where a “fragment” comprises a complementary pair of forward and reverse sequence reads.

In some instances, the fragment-level methylation status value may be calculated as the fraction of the total number of CpG sites within each genomic region that are methylated. In some instances, the fragment-level methylation status value may be calculated based on individual sequence read pairs (e.g., complementary pairs of forward and reverse sequence reads) that overlap with all or a portion of a specified genomic region. In some instances, the fragment-level methylation status value may be calculated based on the set of paired sequence reads that collectively overlap a specified genomic region.

At step 506 in FIG. 5, a statistical analysis to compare the fragment-level methylation status values determined for one or more genomic regions of the set of selected “compact” genomic regions in the subject to corresponding fragment-level methylation status values determined for one or more genomic regions of the set of selected genomic regions for a cohort of individuals having the specified disease. In some instances, the statistical analysis may comprise a t-test, a standardized mean difference test, a permutation test, a binned chi-squared test, or a Kolmogorov-Smirnov test.

Alternatively, in some instances a methylation fraction value may be determined for each CpG locus in each fragment (e.g., pair of forward and reverse sequence reads) at step 504, and a statistical analysis (e.g., a Binomial test) may be performed at step 506 to compare the methylation status of a fragment under test (FUT) for the subject to the methylation status of fragments from diseased individuals in the same compact genomic region. For example, assume that each fragment comprises N CpG loci and that m of them are methylated. One can collect all of the fragments for a specified compact genomic region in samples from a cohort of diseased individuals, sum up all values of N and m, and compute a probability that a given locus is methylated in the compact genomic region in diseased subjects as P=Σᵢ(mᵢ)/Σᵢ(Nᵢ). In some instances, for example, one can then can take a given fragment for a sample from a subject of unknown disease status and run a Binomial test based on (m, N, P) to determine how likely is it that the given fragment came from the distribution of diseased subject fragments. In some instances, one can use Kullbeck-Liebler divergence to perform this fragment assessment as m and N are discrete values.

In some instances, one may compute fragment-level methylation status values and compare their distributions for an unknown sample and a set of samples from a cohort of diseased patients using, e.g., a Kolmogorov-Smirnov test.

In some instance, the method may further comprise use of a Kullbeck-Liebler divergence or informatics entropy-based approach to evaluate the difference between the distribution of fragment-level methylation status values determined for the subject and the distribution of fragment-level methylation status values determined for the cohort of diseased individuals.

At step 508 in FIG. 5, a likelihood that the subject has a disease is determined based on the statistical analysis. For example, in some instances, a difference between the distribution of fragment-level methylation status values determined for the subject and the distribution of fragment-level methylation status values determined for the cohort of individuals having the disease is indicative of an absence of the first disease in the subject. In some instances, a similarity between the distribution of fragment-level methylation status values determined for the subject and the distribution of fragment-level methylation status values determined for the cohort of individuals having the disease is indicative of a presence of the disease in the subject.

In some instances, the method may further comprise comparing the difference between the distribution of fragment-level methylation status values determined for the subject and the distribution of fragment-level methylation status values determined for the cohort of diseased individuals to a predetermined threshold. In some instances, if the difference is greater than or equal to the predetermined threshold, a disease-negative status may be output. In some instances, if the difference is less than the predetermined threshold, a disease-positive status may be output.

In some instances, the difference between the distribution of fragment-level methylation status values determined for the subject and the distribution of fragment-level methylation status values determined for the cohort of diseased individuals may comprise a difference in a mean, median, mode, 10th percentile, 25th percentile, 75th percentile, or 90th percentile of the fragment-level methylation status values.

In some instances, the method may further comprise: determining a fragment-level methylation status value for one or more genomic regions of a set of selected “compact”′ genomic regions based on the sequence read data, wherein each selected “compact” genomic region of the set comprises a plurality of CpG sites that are proximal to each other and that exhibit a consistent methylation status in sequence read data for a second cohort of diseased individuals, e.g., individuals diagnosed with a second specified disease such as a second cancer; and performing a statistical analysis (e.g., the same statistical analysis used with the first cohort of diseased individuals or a different statistical analysis) to compare the distribution of the fragment-level methylation status values determined for the one or more genomic regions of the set of selected “compact” genomic regions in the subject to a corresponding distribution of fragment-level methylation status values determined for one or more genomic regions of the set of selected “compact” genomic regions for the second cohort of diseased individuals, where a difference between the distribution of methylation fraction values determined for the subject and the distribution of methylation fraction values determined for the second cohort of diseased individuals is indicative of an absence of the second disease in the subject, or where a similarity between the distribution of methylation fraction values determined for the subject and the distribution of methylation fraction values determined for the second cohort of diseased individuals is indicative of a presence of the second disease in the subject.

In some instances, the method may be repeated for a third, fourth, fifth disease, etc., using data for a third, fourth, fifth, etc., cohort of diseased individuals.

For any of the methods illustrated by the flowcharts depicted in FIG. 2-FIG. 5, the set of selected “compact” genomic regions may be selected based on the subject's age, sex, race, body mass index, smoking history, clinical history, family history, genetic predisposition for disease, diagnosed disease, polygenic risk score, or any combination thereof.

For any of the methods illustrated by the flowcharts depicted in FIG. 2-FIG. 5, the set of selected genomic regions (e.g., “compact” genomic regions) may comprise at least 5, at least 10, at least 20, at least 30, at least 40, at least 50, at least 60, at least 70, at least 80, at least 90, at least 100, at least 200, at least 400, at least 600, at least 800, at least 1,000, at least 2,000, at least 4,000, at least 6,000, at least 8,000, at least 10,000, at least 20,000, at least 30,000, at least 40,000, at least 50,000, at least 60,000, at least 70,000, at least 80,000, at least 90,000, or at least 100,000 genomic regions.

For any of the methods illustrated by the flowcharts depicted in FIG. 2-FIG. 5, each selected “compact” genomic region may comprise at least N CpG sites within a sequence of L bases in length. In some instances, for example, N may be 2, 3, 4, 5, 6, 7, 8, 9, 10, or more than 10. In some instances, L may be 50, 100, 150, 200, 250, 300, 350, 400, 450, 500, or more than 500 bases.

In some instances of any of the methods illustrated by the flowcharts depicted in FIG. 2 FIG. 5, the methylation fraction values or fragment-level methylation fraction status values determined for the subject may be used as input for a machine learning model configured to output a prediction of a probability or likelihood that the subject has the disease. In some instances, the machine learning model may comprise, for example, a supervised machine learning model. In some instances, the supervised machine learning model may comprise, for example, a linear regression, random forest, support vector machine, artificial neural network or deep learning model.

In some instances, the machine learning model may be trained using a dataset comprising methylation fraction value data or fragment-level methylation fraction status value data for a cohort of subjects diagnosed with a disease and/or for a cohort of healthy individuals.

For any of the methods illustrated by the flowcharts depicted in FIG. 2-FIG. 5, the sample from the subject may comprise a tissue biopsy sample, a liquid biopsy sample, a cervical swab sample, a pap smear samples, or a normal control. In some instances, the sample may be a liquid biopsy sample and comprises blood, plasma, cerebrospinal fluid, sputum, stool, urine, or saliva. In some instances, the sample may be a liquid biopsy sample and comprises circulating tumor cells (CTCs). In some instances, the sample may be a liquid biopsy sample and comprises cell-free DNA (cfDNA), circulating tumor DNA (ctDNA), or any combination thereof. In some instances, the sample may be a cervical swab or Pap smear sample and comprises cells from the subject's cervix.

For any of the methods illustrated by the flowcharts depicted in FIG. 2-FIG. 5, the methylation fraction value for each CpG site or the fragment-level methylation status value for each genomic region may be determined based on sequence read data obtained using a nucleic acid sequencing method comprising the use of a bisulfite- or enzymatic-conversion reaction to convert non-methylated cytosine to uracil (see, e.g., Li, et al. (2011), “DNA Methylation Detection: Bisulfite Genomic Sequencing Analysis”, Methods Mol. Biol. 791:11-21).

For any of the methods illustrated by the flowcharts depicted in FIG. 2-FIG. 5, the methylation fraction value for each CpG site or the fragment-level methylation status value for each genomic region may be determined based on sequence read data obtained using a nucleic acid sequencing method comprising the use of alternative chemical and/or enzymatic reactions may be utilized to convert non-methylated cytosine to uracil (or to convert methylated cytosine to uracil), for example, enzymatic deamination of non-methylated cytosine using APOBEC to form uracil (see, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

For any of the methods illustrated by the flowcharts depicted in FIG. 2-FIG. 5, the methylation fraction value for each CpG site or the fragment-level methylation status value for each genomic region may be determined based on sequence read data obtained using a targeted nucleic acid sequencing method that utilizes proteins comprising methyl-binding domains to capture genomic regions comprising methylated CpG sites.

For any of the methods illustrated by the flowcharts depicted in FIG. 2-FIG. 5, the disease, first disease, or second disease, etc., may be a cancer.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert non-methylated cytosine (or methylated cytosine) to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (viii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods may be used to select a subject (e.g., a patient) for a clinical trial based on the distribution of methylation fraction values or fragment-level methylation status values determined for the subject through the evaluation of methylation status in one or more “compact” genomic regions. In some instances, patient selection for clinical trials based on, e.g., the distribution of methylation fraction values or fragment-level methylation status values determined through the evaluation of methylation status in one or more “compact” genomic regions, may accelerate the development of targeted therapies and improve the healthcare outcomes for treatment decisions.

In some instances, the disclosed methods for determining a distribution of methylation fraction values or fragment-level methylation status values based on the evaluation of methylation status in one or more “compact” genomic regions may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwle (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozolc (Femara), lisocabtagenc maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pcmigatinib (Pcmazyrc), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for determining a distribution of methylation fraction values or fragment-level methylation status values based on the evaluation of methylation status in one or more “compact” genomic regions may be used in treating a disease (e.g., a cancer) in a subject. For example, in response to determining a distribution of methylation fraction values or fragment-level methylation status values using any of the methods disclosed herein, an effective amount of an anti-cancer therapy or anti-cancer treatment may be administered to the subject.

In some instances, the disclosed methods for determining a distribution of methylation fraction values or fragment-level methylation status values based on the evaluation of methylation status in one or more “compact” genomic regions may be used for monitoring discase progression or recurrence (e.g., cancer or tumor progression or recurrence) in a subject. For example, in some instances, the methods may be used to determine a distribution of methylation fraction values or fragment-level methylation status values in a first sample obtained from the subject at a first time point, and used to determine a distribution of methylation fraction values or fragment-level methylation status values in a second sample obtained from the subject at a second time point, where comparison of the first determination of the distribution and the second determination of the distribution allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the distribution of methylation fraction values or fragment-level methylation status values.

In some instances, the methylation fraction values or fragment-level methylation status values, or changes in the distribution thereof relative to a corresponding distribution for a cohort of healthy individuals and/or a cohort of diseased individuals determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining a distribution of methylation fraction values or fragment-level methylation status values based on the evaluation of methylation status in one or more “compact” genomic regions may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining a distribution of methylation fraction values or fragment-level methylation status values based on the evaluation of methylation status in one or more “compact” genomic regions as part of a genomic profiling process (or inclusion of the output from the disclosed methods for determining a distribution of methylation fraction values or fragment-level methylation status values based on the evaluation of methylation status in one or more “compact” genomic regions as part of the genomic profile of the subject) can improve the validity of, e.g., disease detection calls and treatment decisions, made on the basis of the genomic profile by, for example, independently confirming the methylation status of genomic DNA in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of less than 0.1%, less than 1%, at least 0.1-20%, at least 1-25%, at least 5-50%, at least 10-40%, at least 15-25%, or at least 20-30% tumor cell nuclei. In some instances, the sample may comprise a tumor content of less than 0.1%, at least 0.1%, at last 1%, at least 2%, at least 3%, at least 4%, at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM 1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids arc fragmented using any of the methods described above, optionally subjected to repair of chain end damage, optionally ligated to synthetic adapters, primers, and/or barcodes (e.g., amplification primers, sequencing adapters, flow cell adapters, substrate adapters, sample barcodes or indexes, and/or unique molecular identifier sequences), optionally subjected to a chemical or enzymatic reaction to convert non-methylated cytosine to uracil (or to convert methylated cytosine to uracil using alternative chemistries), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or nonspecific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, 5%, 4%, 3%, 2%, 1%, or 0.1% of the genomic DNA. In some instances, the library may include less than 0.1%, 0.01%, 0.001%, 0.0001%, 0.00001%, 0.000001%, or 0.0000001% of the genomic DNA. In some instances, the library may consist of cDNA copies of genomic DNA that include copies of at least 95%, 90%, 80%, 70%, 60%, 50%, 40%, 30%, 20%, 10%, 5%, 4%, 3%, 2%, 1%, or 0.1% of the genomic DNA. In some instances, the library may consist of cDNA copies of genomic DNA that include copies of less than 0.1%, 0.01%, 0.001%, 0.0001%, 0.00001%, 0.000001%, or 0.0000001% of the genomic DNA. In some instances, the library may comprise DNA fragments that have been enriched for different methylation states. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval may be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500 ×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite- or enzymatic-conversion reaction to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions may be utilized to convert non-methylated cytosine to uracil (or to convert methylated cytosine to uracil), for example, enzymatic deamination of non-methylated cytosine using APOBEC to form uracil (see, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27(11):1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11):1571-1572), TARGOMICS (Garinet, et al. (2017), “Calling Chromosome Alterations, DNA Methylation Statuses, and Mutations in Tumors by Simple Targeted Next-Generation Sequencing-A Solution for Transferring Integrated Pangenomic Studies into Routine Practice?”, J. Molecular Diagnostics 19(5):776-787), Bicycle (Grana, et al. (2018) “Bicycle: A Bioinformatics Pipeline to Analyze Bisulfite Sequencing Data”, Bioinformatics 34(8):1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for detecting disease or determining a likelihood that a disease in present in a subject based on an analysis of the DNA methylation status of a selected set of “compact” genomic regions in a sample from the subject. The systems may comprise, e.g., one or more processors, and a memory unit communicatively coupled to the one or more processors and configured to store instructions that, when executed by the one or more processors, cause the system to, for example, receive sequence read data for a plurality of sequence reads derived from a sample from the subject; determine a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and perform a statistical analysis to compare a distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the plurality of healthy individuals, wherein a difference between the distribution of methylation fraction values determined for the subject and the distribution of methylation fraction values determined for the plurality of healthy individuals is indicative of a presence of disease in the subject.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for detecting disease or determining a likelihood that a disease in present in a subject based on an analysis of the DNA methylation status of a selected set of “compact” genomic regions in any of a variety of samples as described herein (e.g., a tissue sample, biopsy sample, hematological sample, or liquid biopsy sample derived from the subject).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a methylation fraction value and/or fragment-level methylation status value for one or more genomic regions from a selected set of “compact” genomic regions, or changes in a distribution thereof may be used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

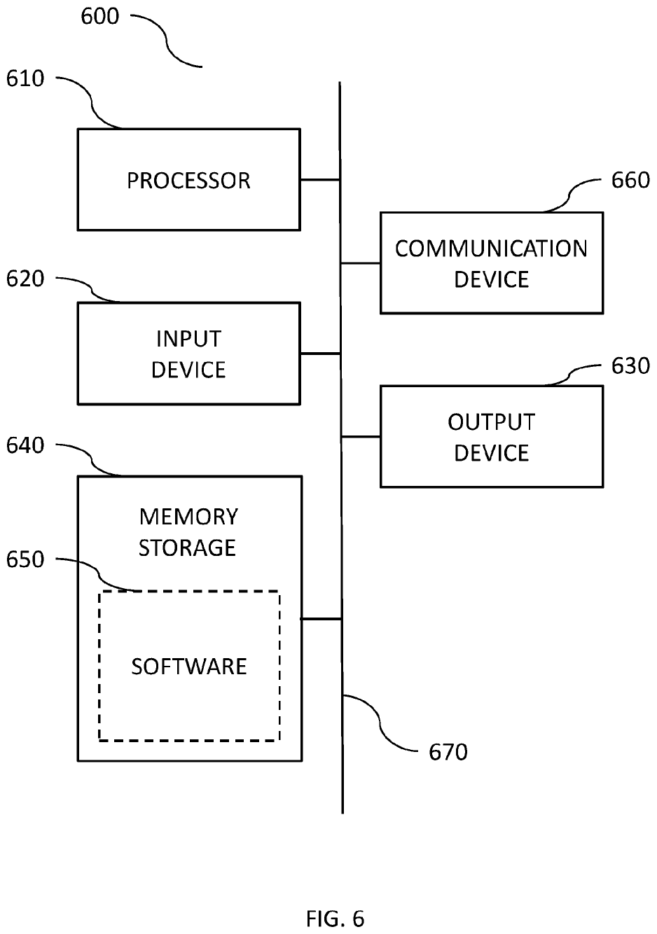

FIG. 6 illustrates an example of a computing device or system in accordance with one embodiment. Device 600 can be a host computer connected to a network. Device 600 can be a client computer or a server. As shown in FIG. 6, device 600 can be any suitable type of microprocessor-based device, such as a personal computer, workstation, server or handheld computing device (portable electronic device) such as a phone or tablet. The device can include, for example, one or more processor(s) 610, input devices 620, output devices 630, memory or storage devices 640, communication devices 660, and nucleic acid sequencers 670. Software 650 residing in memory or storage device 640 may comprise, e.g., an operating system as well as software for executing the methods described herein. Input device 620 and output device 630 can generally correspond to those described herein, and can either be connectable or integrated with the computer.

Input device 620 can be any suitable device that provides input, such as a touch screen, keyboard or keypad, mouse, or voice-recognition device. Output device 630 can be any suitable device that provides output, such as a touch screen, haptics device, or speaker.

Storage 640 can be any suitable device that provides storage (e.g., an electrical, magnetic or optical memory including a RAM (volatile and non-volatile), cache, hard drive, or removable storage disk). Communication device 660 can include any suitable device capable of transmitting and receiving signals over a network, such as a network interface chip or device. The components of the computer can be connected in any suitable manner, such as via a wired media (e.g., a physical system bus 680, Ethernet connection, or any other wire transfer technology) or wirelessly (e.g., Bluetooth®, Wi-Fi®, or any other wireless technology).

Software module 650, which can be stored as executable instructions in storage 640 and executed by processor(s) 610, can include, for example, an operating system and/or the processes that embody the functionality of the methods of the present disclosure (e.g., as embodied in the devices as described herein).

Software module 650 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 640, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

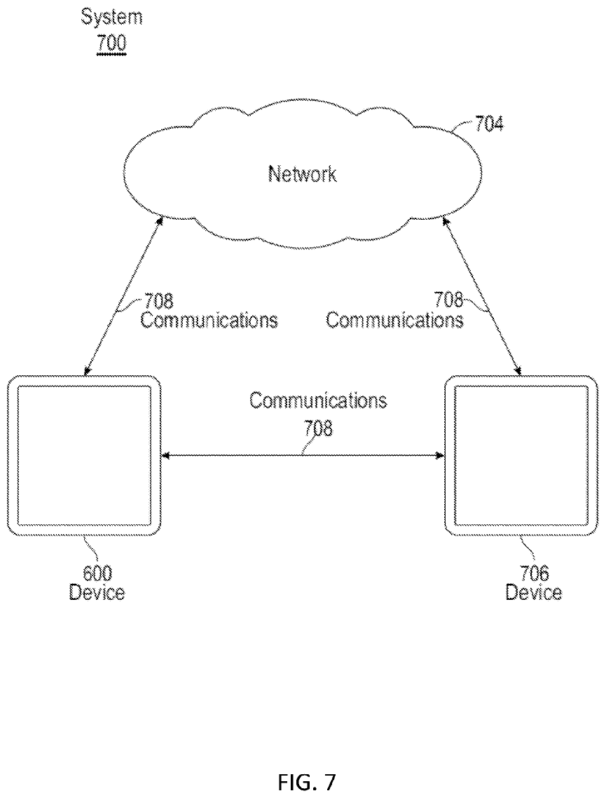

Device 600 may be connected to a network (e.g., network 704, as shown in FIG. 7 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 600 can be implemented using any operating system, e.g., an operating system suitable for operating on the network. Software module 650 can be written in any suitable programming language, such as R, C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 610.

Device 600 can further include a sequencer 670, which can be any suitable nucleic acid sequencing instrument.

FIG. 7 illustrates an example of a computing system in accordance with one embodiment. In system 700, device 600 (e.g., as described above and illustrated in FIG. 6) is connected to network 704, which is also connected to device 706. In some embodiments, device 706 is a sequencer. Exemplary sequencers can include, without limitation, Roche/454's Genome Sequencer (GS) FLX System, Illumina/Solexa's Genome Analyzer (GA), Illumina's HiSeq®2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 600 and 706 may communicate, e.g., using suitable communication interfaces via network 704, such as a Local Area Network (LAN), Virtual Private Network (VPN), or the Internet. In some embodiments, network 704 can be, for example, the Internet, an intranet, a virtual private network, a cloud network, a wired network, or a wireless network. Devices 600 and 706 may communicate, in part or in whole, via wireless or hardwired communications, such as Ethernet, IEEE 802.11b wireless, or the like. Additionally, devices 600 and 706 may communicate, e.g., using suitable communication interfaces, via a second network, such as a mobile/cellular network. Communication between devices 600 and 706 may further include or communicate with various servers such as a mail server, mobile server, media server, telephone server, and the like. In some embodiments, Devices 600 and 706 can communicate directly (instead of, or in addition to, communicating via network 704), e.g., via wireless or hardwired communications, such as Ethernet, IEEE 802.11b wireless, or the like. In some embodiments, devices 600 and 706 communicate via communications 708, which can be a direct connection or can occur via a network (e.g., network 704).

One or all of devices 600 and 706 generally include logic (e.g., http web server logic) or are programmed to format data, accessed from local or remote databases or other sources of data and content, for providing and/or receiving information via network 704 according to various examples described herein.

### EXAMPLES

### Example 1—“Compact” Genomic Regions & Their Utility

The methylation status of CpG loci in the genome can be perturbed in early stage cancer, thus detection of these perturbations may provide a means to detect cancer early in its development. However, accurate detection of these sometimes small perturbations can be hindered by the variability in DNA methylation levels that arises during DNA replication and cell division in vivo, as well as noise in the in vitro techniques used to detect DNA methylation status.

The methylation statuses of CpG loci in the genome of healthy individuals are often not independent, but rather are influenced by the local topology of CpG loci to have a similar methylation status (see, e.g., Lövkvist, et al. (2016), “DNA Methylation in Human Epigenomes Depends on Local Topology of CpG Sites”, Nucleic Acids Research 44(11):5123-5132). We have observed that this cooperative group behavior in some genomic regions in healthy samples can break down in cancer.

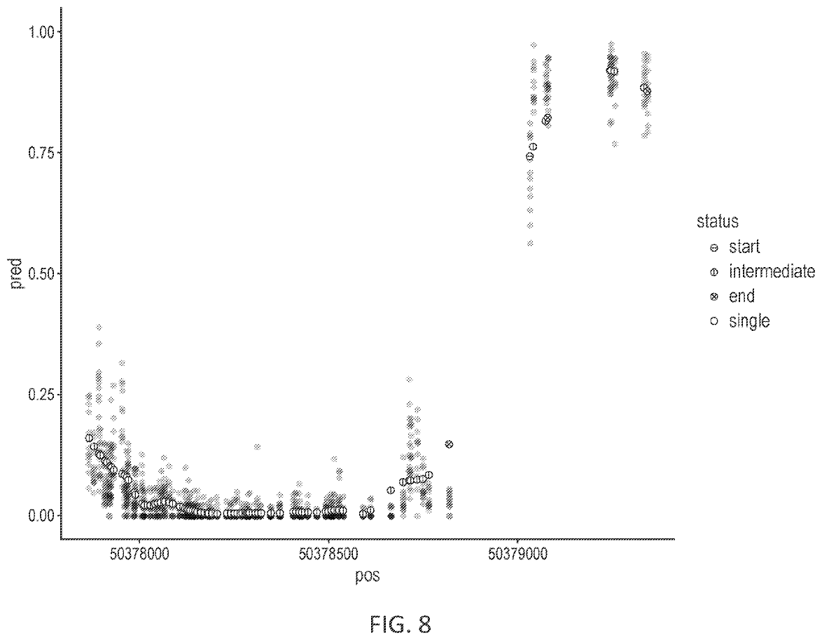

FIG. 8 provides a non-limiting example of a plot of CpG loci methylation fraction values (raw and fitted) in healthy individuals (i.e., individuals not diagnosed as having cancer) as a function of genomic position in lung tissue DNA. The genomic region shown in the plot comprises about 96 CpG sites located within a stretch of sequence of about 1500-1700 bp in length. As can be seen, the per-locus methylation fraction values are fairly consistent over a range of genomic positions spanning from about 50377800 to about 50378750, and very consistent over the range of genomic positions spanning from 50378000 to 50378500. As indicated in the figure legend, the color-coded methylation fraction values for each locus that were derived from a fit of the raw data to a LOESS model (LOESS range threshold=0.15) and then labeled as “start”, “intermediate”, “end”, or “single” CpG loci as determined using an iterative process for identifying “compact” genomic regions (d=100 bases) as described elsewhere herein.

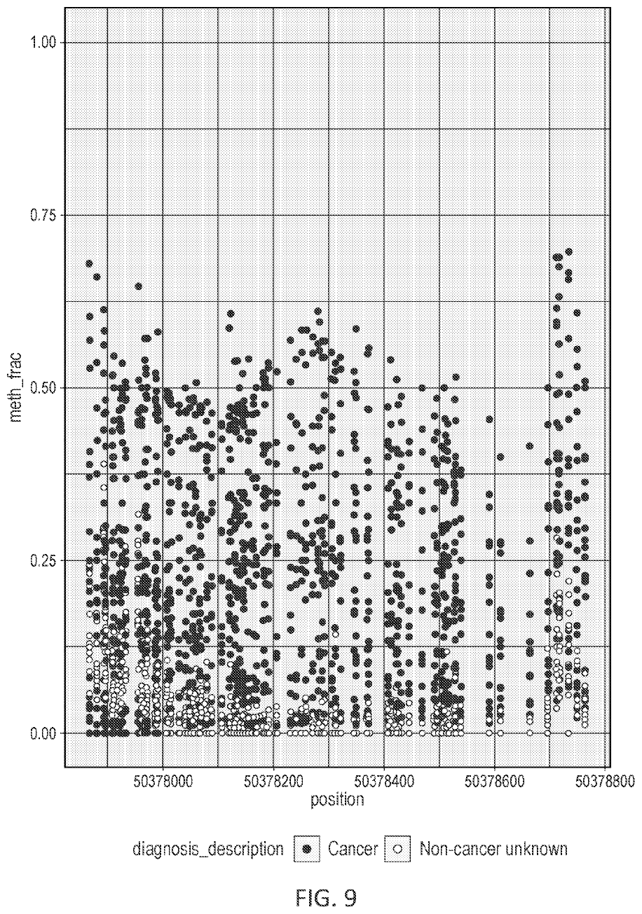

FIG. 9 provides a non-limiting example of a plot of CpG loci methylation fraction values for both cancer patients and healthy individuals as a function of genomic position in lung tissue DNA for a portion of the genomic region illustrated in FIG. 8 (i.e., the genomic sub-region at the lower left of FIG. 8). As can be seen, there is much greater variability in the methylation fraction values for CpG loci in DNA extracted from samples from diseased individuals.

We define “compact” regions as contiguous groups of CpG loci that are in reasonably close physical proximity to each other and that have similar methylation fraction values among healthy subjects (or among a cohort of patients diagnoses with a specified disease, e.g., a cancer, in some instances of the disclosed methods) when evaluating samples of a uniform type (e.g., lung tissue, colon tissue, plasma, etc.).

The key advantage of evaluating methylation status for one or more “compact” genomic regions for use in, e.g., cancer detection is that they enable sensitive and specific statistical tests to address the question: “Is it likely that this unknown sample exhibits methylation status data that is similar to that for a set of known samples from healthy individuals?”.

The close physical proximity of groups of CpG loci within a “compact” genomic region helps to ensure that similar levels of methylation will be observed in the healthy samples due to the biological functions of the groups of CpG loci and the biological means for maintaining methylation states in genomic DNA. The similar methylation fraction values observed among the groups of CpG loci provide greater sensitivity when testing for whether an unknown sample is different from the healthy samples.

Both per-locus methylation fraction values within a given genomic region, or fragment-level methylation status values for a fragment that maps to the region can serve as useful metrics of methylation status. For assessment of changes in per-loci methylation fraction values, one can use a test for differences in continuous distributions, such as the Kolmogorov-Smirnov test.

For assessment of fragment-level methylation states, one can use a Binomial test or entropy-based approach such as Kullback-Liebler Divergence.

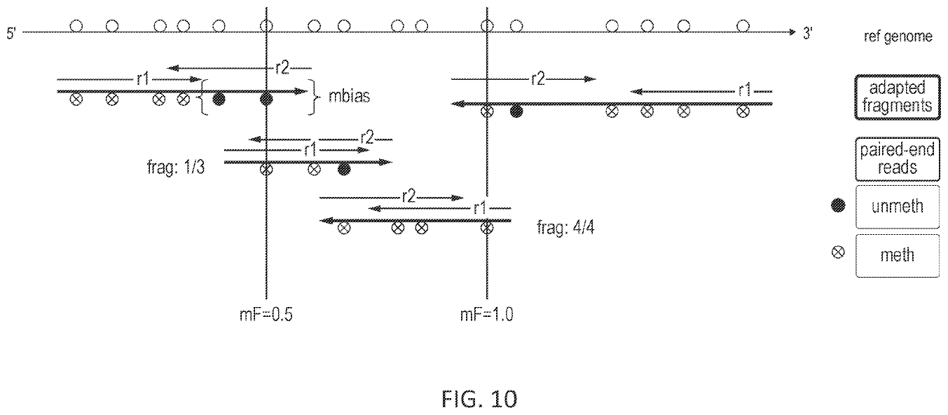

FIG. 10 provides a non-limiting schematic illustration of the calculation of CpG loci methylation fraction values or DNA fragment-level methylation status values for genomic regions (or genomic sub-regions) based on sequence read data.

As indicated in the figure, the methylation fraction value for each CpG site (i.e., a locus-specific methylation fraction value) may be calculated as the fraction of sequence reads covering the given CpG site for which the CpG site is methylated. For example, for the sixth CpG site (indicated by an open circle) from the 5′ end of the reference genome sequence, a first paired sequence read (corresponding to a first pair of forward and reverse sequence reads for a DNA fragment) indicates that the CpG site is methylated (indicated by a circle with an overlapping “x”), and a second paired sequence read (corresponding to a second pair of forward and reverse sequence reads for the DNA fragment) indicates that that the CpG site is un-methylated (solid blue circle), which corresponds to a per-locus methylation fraction value of 0.5.

As indicated in the figure, the fragment-level methylation status value may be calculated as the fraction of the total number of CpG sites within each genomic region that are methylated. For example, the fragment corresponding to the second paired sequence read discussed above comprises one methylated CpG site and two un-methylated CpG sites, for a fragment-level methylation status value of ⅓=0.33. In some instances, the fragment-level methylation status value may be calculated simply as the number of methylated CpG sites in a given fragment. In some instances, the fragment-level methylation status value may be calculated simply as the number of un-methylated CpG sites in a given fragment. In some instances, the fragment-level methylation status value may be calculated as any metric that is a function of the number of methylated CpG sites, the number of un-methylated CpG sites, the total fragment length, or any combination thereof for a given fragment.

In FIG. 10, “mbias” indicates a technical error in the methylation data that may occur with double-stranded library preparation.

### Example 2—Identification of Informative “Compact” Genomic Regions

This non-limiting example describes one approach that may be used for the identification of useful—or informative—“compact” genomic regions. The process starts with determining DNA methylation fraction (mF) values for a set of candidate genomic regions taken from, e.g., the literature or genomic annotations for samples of the same sample type from a plurality of individuals, e.g., healthy individuals or diseased individuals.

The mF values for the CpGs and their corresponding genomic positions may then be fit to a local regression model (e.g., a LOESS model) for that genomic region. Using the CpG genomic position and LOESS-fitted value of mF, and starting at the 5′-end of a candidate genomic region, one performs the following iterative series of steps for each CpG locus in succession to potentially subdivide the candidate genomic region into two or more genomic sub-regions:
If the next CpG site is more than a specified number of base pairs, d, from the current CpG site then the next CpG is assigned to a different genomic sub-region,
If the next CpG site is less than or equal to the specified number of base pairs, d, from the current CpG site, examine the LOESS-fitted mF value of the next CpG site,
If the LOESS-fitted mF value of the next CpG site is such that including the next CpG site in the current genomic sub-region would create a difference of max(mF)−min(mF)>M for all the mF values in that sub-region, then the next CpG is assigned to a different genomic sub-region and the current genomic sub-region is terminated,
If neither of these conditions is exceeded, then the next CpG site is included in the current genomic sub-region and the algorithm proceeds to evaluate the subsequent CpG site,
Repeat as necessary until all CpG sites for the candidate region are evaluated.

CpG loci may then be assigned a label with respect to their position within a given genomic sub-region. For example, CpG loci at the leftmost position (5′-end) of a candidate region, and at the beginning of a new genomic sub-region are assigned the label “start”. CpG loci are assigned the label “end” when the next CpG locus has been allocated to a new genomic sub-region or when the end of the original candidate genomic region is reached. CpG loci positioned between “start” and “end” locations are assigned the label “intermediate”. If the change in LOESS-fitted mF values and/or distance exceeds the above thresholds for two consecutive CpG loci, the CpG position is labeled as “single”. For example, if CpG 10 is part of the current genomic sub-region, and the position of CpG 11 exceeds the distance threshold, d, from the position CpG 10, then CpG 10 is marked as an “end” locus and CpG 11 would be considered the candidate “start” of a new genomic sub-region. However if CpG 12 is positioned at a distance greater than d from CpG11, or including it in the new genomic sub-region would create a difference of max(mF)−min(mF)>M, then CpG 12 would need to be assigned to another new genomic sub-region, different from that for CpG11, and CpG11 would be marked as an “end”. However, since a given CpG site can't have a label of both “start” and “end”, CpG 11 would be relabeled as a “single”, CpG 12 would be labeled as a candidate “start”, and the next CpG site (CpG 13) would then be evaluated.

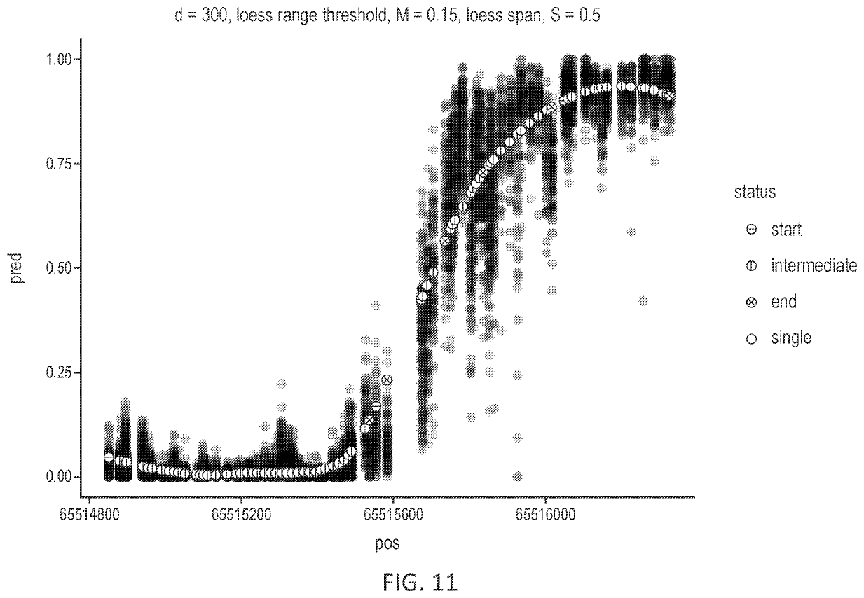

FIG. 11 provides a non-limiting example of a plot of fitted CpG loci methylation fraction values as a function of genomic position that illustrates the subdivision of a candidate genomic region to identify a “compact” genomic region. Methylation fraction (mF) values for CpG loci (partially transparent black dots) were collected for a cohort of healthy individual plasma samples in the genomic window of chromosome 12 shown in the figure.

The LOESS-fitted values for each CpG position are also displayed on the plot (opaque colored dots). The CpG label “status” is represented by color of the LOESS-fitted values (see figure legend).

Sub-region coordinates are defined by the genomic position of “start” and “end” labeled CpG loci, extended by a pad of several bp to either side. CpG loci labeled “single” (green) are removed from further analysis if present.

The iterative evaluation and CpG locus assignment process can be performed using varying values of d and M, which in some instances may range from about 100 to 300 bp and from about 0.1 to 0.15, respectively. For the plot shown in FIG. 11, values of d=300 bp and M=0.15 (the Loess range parameter) were used. The span parameter used in the LOESS model (which controls the amount of smoothing) was 0.5.

Once the refined “compact” genomic region boundaries are defined (e.g., the boundaries of a sub-region of the original candidate genomic region), we assess the methylation status results for cancer specimens in these “compact” genomic regions to refine the set of “compact” genomic regions to identify those that are also informative in differentiating cancer from healthy subjects.

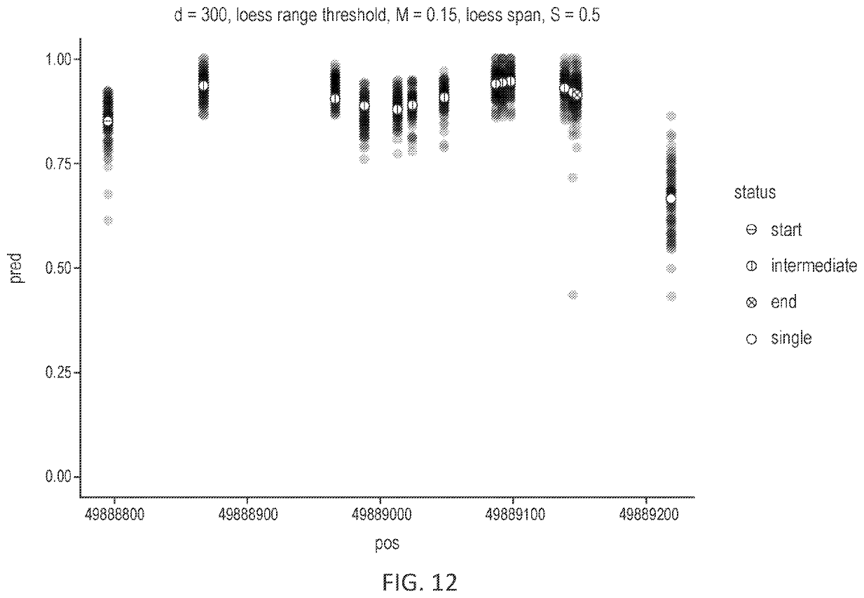

FIG. 12 provides another non-limiting example of using the described process to to identify a “compact” genomic region from a candidate genomic region from chromosome 8. For the plot shown in FIG. 12, values of d=300 bp and M=0.15 were used. The LOESS range threshold (span) was 0.5.

### Exemplary Implementations

Exemplary implementations of the methods and systems described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, sequence read data for the plurality of sequence reads;
   determining, using the one or more processors, a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and
   performing, using the one or more processors, a statistical analysis to compare a distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the plurality of healthy individuals,
   wherein a difference between the distribution of methylation fraction values determined for the subject and the distribution of methylation fraction values determined for the plurality of healthy individuals is indicative of a presence of disease in the subject.
2. The method of clause 1, further comprising comparing, using the one or more processors, the difference between the distribution of methylation fraction values determined for the subject and the distribution of methylation fraction values determined for the plurality of healthy individuals to a first predetermined threshold.
3. The method of clause 2, wherein if the difference is greater than or equal to the first predetermined threshold, a disease-positive status is output by the one or more processors.
4. The method of clause 2, wherein if the difference is less than the first predetermined threshold, a disease-negative status is output by the one or more processors.
5. The method of any one of clauses 1 to 4, wherein the statistical analysis comprises a t-test, a standardized mean difference test, a permutation test, a binned chi-squared test, or a Kolmogorov-Smirnov test.
6. The method of any one of clauses 1 to 5, further comprising use of a Kullbeck-Liebler divergence or informatics entropy-based approach to evaluate a difference between the distribution of methylation fraction values determined for the subject and the distribution of methylation fraction values determined for the plurality of healthy individuals.
7. The method of any one of clauses 1 to 6, wherein the subject is suspected of having or is determined to have cancer.
8. The method of clause 7, wherein the cancer is comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
9. The method of clause 7 or clause 8, further comprising treating the subject with an anti-cancer therapy.
10. The method of clause 9, wherein the anti-cancer therapy comprises a targeted anti-cancer therapy.
11. The method of clause 10, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
12. The method of any one of clauses 1 to 11, further comprising obtaining the sample from the subject.
13. The method of any one of clauses 1 to 12, wherein the sample comprises a tissue biopsy sample, a liquid biopsy sample, or a normal control.
14. The method of clause 13, wherein the sample is a liquid biopsy sample and comprises blood, plasma, cerebrospinal fluid, sputum, stool, urine, or saliva.
15. The method of clause 13, wherein the sample is a liquid biopsy sample and comprises circulating tumor cells (CTCs).
16. The method of clause 13, wherein the sample is a liquid biopsy sample and comprises cell-free DNA (cfDNA), circulating tumor DNA (ctDNA), or any combination thereof.
17. The method of any one of clauses 1 to 16, wherein the plurality of nucleic acid molecules comprises a mixture of tumor nucleic acid molecules and non-tumor nucleic acid molecules.
18. The method of clause 17, wherein the tumor nucleic acid molecules are derived from a tumor portion of a heterogeneous tissue biopsy sample, and the non-tumor nucleic acid molecules are derived from a normal portion of the heterogeneous tissue biopsy sample.
19. The method of clause 18, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
20. The method of any one of clauses 1 to 19, wherein the one or more adapters comprise amplification primers, flow cell adaptor sequences, substrate adapter sequences, or sample index sequences.
21. The method of any one of clauses 1 to 20, wherein the captured nucleic acid molecules are captured from the amplified nucleic acid molecules by hybridization to one or more bait molecules.
22. The method of clause 21, wherein the one or more bait molecules comprise one or more nucleic acid molecules, each comprising a region that is complementary to a region of a captured nucleic acid molecule.
23. The method of any one of clauses 1 to 22, wherein amplifying nucleic acid molecules comprises performing a polymerase chain reaction (PCR) amplification technique, a non-PCR amplification technique, or an isothermal amplification technique.
24. The method of any one of clauses 1 to 23, wherein the sequencing comprises use of a massively parallel sequencing (MPS) technique, whole genome sequencing (WGS), whole exome sequencing, targeted sequencing, direct sequencing, or Sanger sequencing technique.
25. The method of clause 24, wherein the sequencing comprises massively parallel sequencing, and the massively parallel sequencing technique comprises next generation sequencing (NGS).
26. The method of any one of clauses 1 to 25, wherein the sequencer comprises a next generation sequencer.
27. The method of any one of clauses 1 to 26, wherein one or more of the plurality of sequencing reads overlap one or more gene loci within one or more subgenomic intervals in the sample.
28. The method of clause 27, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
29. The method of clause 27 or clause 28, wherein the one or more gene loci comprise ABL 1, ACVR1B, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
30. The method of clause 27 or clause 28, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-16, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, VEGFB, or any combination thereof.
31. The method of any one of clauses 1 to 29, further comprising generating, by the one or more processors, a report indicating the detected difference between the distribution of methylation fraction values determined for the subject and the distribution of methylation fraction values determined for the plurality of healthy individuals, or a likelihood that the disease is present in the subject.
32. The method of clause 31, further comprising transmitting the report to a healthcare provider.
33. The method of clause 32, wherein the report is transmitted via a computer network or a peer-to-peer connection.
34. A method for detecting disease in a subject, the method comprising:
   receiving, at one or more processors, sequence read data for a plurality of sequence reads derived from a sample from the subject;
   determining, using the one or more processors, a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and
   performing, using the one or more processors, a statistical analysis to compare a distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the plurality of healthy individuals,
   wherein a difference between the distribution of methylation fraction values determined for the subject and the distribution of methylation fraction values determined for the plurality of healthy individuals is indicative of a presence of disease in the subject.
35. The method of clause 34, further comprising comparing, using the one or more processors, the difference between the distribution of methylation fraction values determined for the subject and the distribution of methylation fraction values determined for the plurality of healthy individuals to a first predetermined threshold.
36. The method of clause 35, wherein if the difference is greater than or equal to the first predetermined threshold, a disease-positive status is output by the one or more processors.
37. The method of clause 35, wherein if the difference is less than the first predetermined threshold, a disease-negative status is output by the one or more processors.
38. The method of any one of clauses 34 to 37, wherein the difference between the distribution of methylation fraction values determined for the subject and the distribution of methylation fraction values determined for the plurality of healthy individuals comprises a difference in a mean, median, mode, 10th percentile, 25th percentile, 75th percentile, or 90th percentile of the methylation fraction values.
39. The method of any one of clauses 34 to 38, wherein the statistical analysis comprises a t-test, a standardized mean difference test, a permutation test, a binned chi-squared test, or a Kolmogorov-Smirnov test.
40. The method of any one of clauses 34 to 39, further comprising use of a Kullbeck-Liebler divergence or informatics entropy-based approach to evaluate a difference between the distribution of methylation fraction values determined for the subject and the distribution of methylation fraction values determined for the plurality of healthy individuals.
41. The method of any one of clauses 34 to 40, wherein the plurality of CpG sites comprises all CpG sites within a given genomic region.
42. A method for detecting disease in a subject, the method comprising:
   receiving, at one or more processors, sequence read data for a plurality of sequence reads derived from a sample from the subject;
   determining, using the one or more processors, a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a first plurality of individuals having a first disease; and
   performing, using the one or more processors, a statistical analysis to compare a distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the first plurality of individuals having the first disease,
   wherein a difference between the distribution of methylation fraction values determined for the subject and the distribution of methylation fraction values determined for the first plurality of individuals having the first disease is indicative of an absence of the first disease in the subject, or
   wherein a similarity between the distribution of methylation fraction values determined for the subject and the distribution of methylation fraction values determined for the first plurality of individuals having the first disease is indicative of a presence of the first disease in the subject.
43. The method of clause 42, further comprising:
   determining, using the one or more processors, a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a second plurality of individuals having a second disease; and
   performing, using the one or more processors, a statistical analysis to compare the distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the second plurality of individuals having the second disease,
   wherein a difference between the distribution of methylation fraction values determined for the subject and the distribution of methylation fraction values determined for the second plurality of individuals having the second disease is indicative of an absence of the second disease in the subject, or
   wherein a similarity between the distribution of methylation fraction values determined for the subject and the distribution of methylation fraction values determined for the second plurality of individuals having the second disease is indicative of a presence of the second disease in the subject.
44. A method for detecting disease in a subject, the method comprising:
   receiving, at one or more processors, sequence read data for a plurality of sequence reads derived from a sample from the subject;
   determining, using the one or more processors, a fragment-level methylation status value for each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and
   performing, using the one or more processors, a statistical analysis to compare the fragment-level methylation status values determined for one or more genomic regions of the set of selected genomic regions in the subject to corresponding fragment-level methylation status values determined for one or more genomic regions of the set of selected genomic regions for the plurality of healthy individuals,
   wherein a difference between the fragment-level methylation fraction status values determined for one or more genomic regions for the subject and the fragment-level methylation fraction status values determined for the corresponding one or more genomic regions for the plurality of healthy individuals is indicative of a presence of disease in the subject.
45. The method of clause 44, further comprising comparing, using the one or more processors, the difference between the fragment-level methylation status values determined for the one or more genomic regions for the subject and the fragment-level methylation fraction status values determined for the corresponding one or more genomic regions for the plurality of healthy individuals to a second predetermined threshold.
46. The method of clause 45, wherein if the difference is greater than or equal to the second predetermined threshold, a disease-positive status is output by the one or more processors.
47. The method of clause 45, wherein if the difference is less than the second predetermined threshold, a disease-negative status is output by the one or more processors.
48. The method of any one of clauses 44 to 47, wherein the difference between the fragment-level methylation fraction status values determined for one or more genomic regions for the subject and the fragment-level methylation fraction status values determined for the corresponding one or more genomic regions for the plurality of healthy individuals comprises a difference in a mean, median, mode, 10th percentile, 25th percentile, 75th percentile, or 90th percentile of the fragment-level methylation status values.
49. The method of any one of clauses 44 to 48, wherein the statistical analysis comprises a t-test, a standardized mean difference test, a permutation test, a binned chi-squared test, or a Kolmogorov-Smirnov test.
50. A method for detecting disease in a subject, the method comprising:
   receiving, at one or more processors, sequence read data for a plurality of sequence reads derived from a sample from the subject;
   determining, using the one or more processors, a fragment-level methylation status value for each genomic region of a set of selected genomic regions based on the sequence read data, wherein each selected genomic region of the set comprises a plurality of CpG sites that are proximal to each other and that exhibit a consistent methylation status in sequence read data for a first plurality of individuals having a first disease; and
   performing, using the one or more processors, a statistical analysis to compare the fragment-level methylation status values determined for one or more genomic regions of the set of selected genomic regions in the subject to corresponding fragment-level methylation status values determined for one or more genomic regions of the set of selected genomic regions for the first plurality of individuals having the first disease,
   wherein a difference between the fragment-level methylation fraction status values determined for one or more genomic regions for the subject and the fragment-level methylation fraction status values determined for the corresponding one or more genomic regions for the first plurality of individuals having the first disease is indicative of an absence of the first disease in the subject, or
   wherein a similarity between the fragment-level methylation fraction status values determined for one or more genomic regions for the subject and the fragment-level methylation fraction status values determined for the corresponding one or more genomic regions for the first plurality of individuals having the first disease is indicative of a presence of the first disease in the subject.
51. The method of clause 50, further comprising:
   determining, using the one or more processors, a fragment-level methylation status value for each genomic region of a set of selected genomic regions based on the sequence read data, wherein each selected genomic region of the set comprises a plurality of CpG sites that are proximal to each other and that exhibit a consistent methylation status in sequence read data for a second plurality of individuals having a second disease; and
   performing, using the one or more processors, a statistical analysis to compare the fragment-level methylation status values determined for the one or more genomic regions of the set of selected genomic regions in the subject to corresponding fragment-level methylation status values determined for one or more genomic regions of the set of selected genomic regions for a second plurality of individuals having a second disease,
   wherein a difference between the fragment-level methylation fraction status values determined for one or more genomic regions for the subject and the fragment-level methylation fraction status values determined for the corresponding one or more genomic regions for the second plurality of individuals having the second disease is indicative of an absence of the second disease in the subject, or
   wherein a similarity between the fragment-level methylation fraction status values determined for one or more genomic regions for the subject and the fragment-level methylation fraction status values determined for the corresponding one or more genomic regions for the second plurality of individuals having the second disease is indicative of a presence of the second disease in the subject.
52. The method of any one of clauses 34 to 51, wherein the set of selected genomic regions is selected based on the subject's age, sex, race, body mass index, smoking history, clinical history, family history, genetic predisposition for disease, diagnosed disease, polygenic risk score, or any combination thereof.
53. The method of any one of clauses 34 to 52, wherein the set of selected genomic regions comprises at least 5, at least 10, at least 20, at least 30, at least 40, at least 50, at least 60, at least 70, at least 80, at least 90, at least 100, at least 200, at least 400, at least 600, at least 800, at least 1,000, at least 2,000, at least 4,000, at least 6,000, at least 8,000, at least 10,000, at least 20,000, at least 30,000, at least 40,000, at least 50,000, at least 60,000, at least 70,000, at least 80,000, at least 90,000, or at least 100,000 genomic regions.
54. The method of any one of clauses 34 to 53, wherein each selected genomic region comprises at least N CpG sites within a sequence of L bases in length.
55. The method of clause 54, wherein N is 3, 4, or 5.
56. The method of clause 54 or clause 55, wherein L is 50, 100, 150, 250, 300, or 350.
57. The method of any one of clauses 34 to 56, wherein the methylation fraction values or fragment-level methylation fraction status values determined for the subject are used as input for a machine learning model configured to output a prediction of a probability that the subject has the disease.
58. The method of clause 57, wherein the machine learning model comprises a supervised machine learning model.
59. The method of clause 58, wherein the supervised machine learning model comprises a linear regression, random forest, support vector machine, artificial neural network or deep learning model.
60. The method of any one of clauses 57 to 59, wherein the machine learning model is trained using a dataset comprising methylation fraction value data or fragment-level methylation fraction status value data for a cohort of subjects diagnosed with the disease and a cohort of healthy individuals.
61. The method of any one of clauses 34 to 60, wherein the sample from the subject comprises a tissue biopsy sample, a liquid biopsy sample, a cervical swab sample, a pap smear samples, or a normal control.
62. The method of clause 61, wherein the sample is a liquid biopsy sample and comprises blood, plasma, cerebrospinal fluid, sputum, stool, urine, or saliva.
63. The method of clause 61, wherein the sample is a liquid biopsy sample and comprises circulating tumor cells (CTCs).
64. The method of clause 61, wherein the sample is a liquid biopsy sample and comprises cell-free DNA (cfDNA), circulating tumor DNA (ctDNA), or any combination thereof.
65. The method of clause 61, wherein the sample is a cervical swab or Pap smear sample and comprises cells from the subject's cervix.
66. The method of any one of clauses 34 to 65, wherein the methylation fraction value for each CpG site or the fragment-level methylation status value for each genomic region is determined based on sequence read data obtained using a nucleic acid sequencing method comprising the use of a bisulfite- or enzymatic-conversion reaction to convert non-methylated cytosine to uracil.
67. The method of any one of clauses 34 to 65, wherein the methylation fraction value for each CpG site or the fragment-level methylation status value for each genomic region is determined based on sequence read data obtained using a nucleic acid sequencing method comprising the use of a bisulfite- or enzymatic-conversion reaction to convert methylated cytosine to uracil.
68. The method of any one of clauses 34 to 65, wherein the methylation fraction value for each CpG site or the fragment-level methylation status value for each genomic region is determined based on sequence read data obtained using a targeted nucleic acid sequencing method that utilizes proteins comprising methyl-binding domains to capture genomic regions comprising methylated CpG sites.
69. The method of any one of clauses 34 to 68, wherein the disease, first disease, or second disease is cancer.
70. A method for identifying informative genomic sub-regions, the method comprising:
   receiving, at one or more processors, sequence read data for a plurality of candidate genomic regions in samples from a plurality of healthy individuals;
   determining, using the one or more processors, a methylation fraction value for a plurality of CpG sites within each of the plurality of candidate genomic regions in each of the plurality of samples from the healthy individuals based on the sequence read data;
   determining, using the one or more processors, a fitted methylation fraction value for a plurality of CpG sites within each candidate genomic region by fitting genomic positions of the CpG sites and corresponding methylation fraction values of the plurality of CpG sites to a local regression model;
   identifying, using the one or more processors, one or more genomic sub-regions for each candidate genomic region by iteratively evaluating sequences of different length that: (i) exhibit a consistent methylation status in sequence read data for a plurality of healthy individuals, and (ii) comprise a plurality of CpG sites that are proximal to each other;
   identifying starting and ending genomic positions for each of the one or more genomic sub-regions; and
   assigning a label to each CpG site within the one or more genomic sub-regions based on their position within the genomic sub-region.
71. The method of clause 70, wherein identifying the one or more genomic sub-regions for each candidate genomic region comprises repeating, for each successive CpG site starting at a 5′-end of each candidate genomic region, the steps of:
   comparing a genomic position and a fitted methylation fraction value for a current CpG site to a genomic position and fitted methylation fraction value for a next CpG site;
   determining a separation distance between the current CpG site and the next CpG site;
   determining a methylation metric calculated based on the fitted methylation fraction values for a current genomic sub-region and
in response to the separation distance being greater than a specified number of bases, d: (1) assigning the next CpG site to a new genomic sub-region, and (2) terminating a current genomic sub-region; or
   in response to the methylation metric being greater than a predetermined value, M, if the next CpG site were included in the current genomic sub-region: (1) assigning the next CpG site to a new genomic sub-region, and (2) terminating the current genomic sub-region; or
   assigning the next CpG site to the current genomic sub-region.
72. The method of clause 70 or clause 71, further comprising assessing methylation fraction value data for the one or more identified genomic sub-regions for samples from a plurality of patients diagnosed with a specified discase to identify genomic sub-regions that may be used to differentiate between patients diagnosed with the disease and healthy individuals.
73. The method of any one of clauses 70 to 72, wherein the plurality of candidate genomic regions are selected from annotated genomic regions identified in a genomics database as exhibiting differential methylation status for a specified disease.
74. The method of any one of clauses 70 to 73, wherein the plurality of candidate genomic regions are selected from genomic regions identified in a scientific publication as exhibiting differential methylation status for a specified disease.
75. The method of any one of clauses 70 to 74, wherein the plurality of candidate genomic regions are selected from genomic regions identified in a genomics database that comprise cell type-specific markers, markers related to transcriptional programs, genes, or additional genomic features that have not been shown to exhibit differential methylation in a specified disease.
76. The method of clause 75, wherein the additional genomic features comprise repeat elements, enhancers, promoters, DNase1 hypersensitive sites (DHSs), or any combination thereof.
77. The method of any one of clauses 70 to 76, wherein the plurality of candidate genomic regions are selected from genomic regions associated with a functional pathway in a specified disease.
78. The method of clause 77, wherein the specified disease is cancer and the genomic regions comprise tumor suppressor genes or oncogenes.
79. The method of clause 77, wherein the specified disease is an immune system disorder and the genomic regions comprise major histocompatibility complex genes.
80. The method of any one of clauses 74 to 79, wherein the specified disease comprises a cancer.
81. The method of any one of clauses 70 to 80, wherein the local regression model comprises a LOESS model.
82. The method of any one of clauses 70 to 80, wherein the local regression model comprises a LOWESS model.
83. The method of any one of clauses 70 to 82, wherein the one or more genomic sub-regions identified comprise at least 5, at least 10, at least 20, at least 30, at least 40, at least 50, at least 60, at least 70, at least 80, at least 90, at least 100, at least 200, at least 400, at least 600, at least 800, at least 1,000, at least 2,000, at least 4,000, at least 6,000, at least 8,000, at least 10,000, at least 20,000, at least 30,000, at least 40,000, at least 50,000, at least 60,000, at least 70,000, at least 80,000, at least 90,000, or at least 100,000 genomic sub-regions.
84. The method of any one of clauses 71 to 83, wherein the specified number of bases, d, ranges from 10 bp to 1,000 bp.
85. The method of any one of clauses 71 to 84, wherein the specified number of bases, d, ranges from 100 bp to 300 bp.
86. The method of any one of clauses 71 to 85, wherein the methylation metric is calculated based on the fitted methylation fraction values for all CpG sites in the current genomic sub-region and the fitted methylation fraction value for the next CpG site.
87. The method of clause 86, wherein the methylation metric is calculated according to the relationship:

methylation metric=max(mF)−min(mF)

for all of the fitted methylation fraction (mF) values for CpG sites in the current sub-region.
88. The method of any one of clauses 71 to 87, wherein the value of M ranges from 0.05 to 0.3
89. The method of any one of clauses 71 to 88, wherein the value of M ranges from 0.1 to 0.15.
90. The method of any one of clauses 71 to 89, wherein each CpG located at the 5′-end of a candidate genomic region or at a 5′-end of a new genomic sub-region is assigned a label of “start”.
91. The method of any one of clauses 71 to 90, wherein each CpG located at a 3′-end of a candidate genomic region or at a 3′-end of a new genomic sub-region is assigned a label of “end”.
92. The method of any one of clauses 71 to 91, wherein each CpG located between a “start” CpG and an “end” CpG for a same genomic sub-region is assigned a label of “intermediate”.
93. The method of any one of clauses 70 to 92, wherein the identified genomic sub-regions are further extended by a short section of additional nucleic acid sequence at either end of the genomic sub-region defined by a corresponding pair of “start” and “end” CpGs.
94. The method of any one of clauses 70 to 93, wherein the samples comprise tissue biopsy samples, liquid biopsy samples, cervical swab samples, pap smear samples, normal controls, or any combination thereof.
95. The method of clause 94, wherein the samples are liquid biopsy samples and comprise blood, plasma, cerebrospinal fluid, sputum, stool, urine, or saliva.
96. The method of clause 94, wherein the samples are liquid biopsy samples and comprise circulating tumor cells (CTCs).
97. The method of clause 94, wherein the samples are liquid biopsy samples and comprise cell-free DNA (cfDNA), circulating tumor DNA (ctDNA), or any combination thereof.
98. The method of clause 94, wherein the samples are cervical swab or Pap smear samples and comprise cells from a cervix in a plurality of healthy individuals.
99. The method of any one of clauses 70 to 98, wherein the methylation fraction values are determined based on sequence read data obtained using a nucleic acid sequencing method comprising the use of a bisulfite- or enzymatic-conversion reaction to convert non-methylated cytosine to uracil.
100. The method of any one of clauses 70 to 98, wherein the methylation fraction values are determined based on sequence read data obtained using a nucleic acid sequencing method comprising the use of a bisulfite- or enzymatic-conversion reaction to convert methylated cytosine to uracil.
101. The method of any one of clauses 70 to 98, wherein the methylation fraction values are determined based on sequence read data obtained using a targeted nucleic acid sequencing method that utilizes proteins comprising methyl-binding domains to capture genomic regions comprising methylated CpG sites.
102. The method of any one of clauses 34 to 69, wherein the determination of a difference between the distribution of methylation fraction values determined for the subject and the distribution of methylation fraction values determined for a plurality of healthy individuals or diseased individuals is used to diagnose or confirm a diagnosis of disease in the subject.
103. The method of clause 102, wherein the disease is cancer.
104. The method of clause 103, wherein the cancer is acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
105. The method of clause 103 or clause 104, further comprising selecting an anti-cancer therapy to administer to the subject based on the determination of a difference between the distribution of methylation fraction values determined for the subject and the distribution of methylation fraction values determined for a plurality of healthy individuals or diseased individuals.
106. The method of clause 105, further comprising determining an effective amount of an anti-cancer therapy to administer to the subject based on the determination of a difference between the distribution of methylation fraction values determined for the subject and the distribution of methylation fraction values determined for a plurality of healthy individuals or diseased individuals.
107. The method of clause 105 or clause 106, further comprising administering the anti-cancer therapy to the subject based on the determination of a difference between the distribution of methylation fraction values determined for the subject and the distribution of methylation fraction values determined for a plurality of healthy individuals or diseased individuals.
108. The method of any one of clauses 105 to 107, wherein the anti-cancer therapy comprises chemotherapy, radiation therapy, immunotherapy, a targeted therapy, or surgery.
109. A method for diagnosing a disease, the method comprising:
   diagnosing that a subject has the disease based on a determination of a difference between the distribution of methylation fraction values determined for a subject and the distribution of methylation fraction values determined for a plurality of healthy individuals or diseased individuals for a sample from the subject, wherein the difference between the distribution of methylation fraction values determined for the subject and the distribution of methylation fraction values determined for a plurality of healthy individuals or diseased individuals is determined according to the method of any one of clauses 34 to 108.
110. A method of selecting an anti-cancer therapy, the method comprising:
   responsive to determining a difference between the distribution of methylation fraction values determined for a sample from a subject and the distribution of methylation fraction values determined for samples from a plurality of healthy individuals or diseased individuals, selecting an anti-cancer therapy for the subject, wherein the difference between the distribution of methylation fraction values determined for the subject and the distribution of methylation fraction values determined for a plurality of healthy individuals or diseased individuals for a sample from the subject is determined according to the method of any one of clauses 34 to 108.
111. A method of treating a cancer in a subject, comprising:
   responsive to determining a difference between the distribution of methylation fraction values determined for a sample from a subject and the distribution of methylation fraction values determined for samples from a plurality of healthy individuals or diseased individuals, administering an effective amount of an anti-cancer therapy to the subject, wherein the difference between the distribution of methylation fraction values determined for the sample from the subject and the distribution of methylation fraction values determined for samples from a plurality of healthy individuals or diseased individuals is determined according to the method of any one of clauses 34 to 108.
112. A method for monitoring cancer progression or recurrence in a subject, the method comprising:
   determining a first difference between the distribution of methylation fraction values determined for a sample from a subject and the distribution of methylation fraction values in samples from a plurality of healthy individuals or diseased individuals at a first time point according to the method of any one of clauses 34 to 108;
   determining a second difference between the distribution of methylation fraction values determined for a sample from the subject and the distribution of methylation fraction values for the plurality of healthy individuals or diseased individuals at a second time point; and comparing the first determination to the second determination, thereby monitoring the cancer progression or recurrence.
113. The method of clause 112, wherein the second determination for the second sample is determined according to the method of any one of clauses 34 to 108.
114. The method of clause 112 or clause 113, further comprising selecting an anti-cancer therapy for the subject in response to the cancer progression.
115. The method of clause 112 or clause 113, further comprising administering an anti-cancer therapy to the subject in response to the cancer progression.
116. The method of clause 112 or clause 113, further comprising adjusting an anti-cancer therapy for the subject in response to the cancer progression.
117. The method of any one of clauses 114 to 116, further comprising adjusting a dosage of the anti-cancer therapy or selecting a different anti-cancer therapy in response to the cancer progression.
118. The method of clause 117, further comprising administering the adjusted anti-cancer therapy to the subject.
119. The method of any one of clauses 112 to 118, wherein the first time point is before the subject has been administered an anti-cancer therapy, and wherein the second time point is after the subject has been administered the anti-cancer therapy.
120. The method of any one of clauses 112 to 119, wherein the subject has a cancer, is at risk of having a cancer, is being routine tested for cancer, or is suspected of having a cancer.
121. The method of any one of clauses 112 to 120, wherein the cancer is a solid tumor.
122. The method of any one of clauses 112 to 120, wherein the cancer is a hematological cancer.
123. The method of any one of clauses 114 to 122, wherein the anti-cancer therapy comprises chemotherapy, radiation therapy, immunotherapy, a targeted therapy, or surgery.
124. The method of any one of clauses 34 to 69, further comprising determining, identifying, or applying a value for the difference between the distribution of methylation fraction values determined for a sample from a subject and the distribution of methylation fraction values in samples from a plurality of healthy individuals or diseased individuals as a diagnostic value associated with the sample from the subject.
125. The method of any one of clauses 34 to 69, further comprising generating a genomic profile for the subject based on the determination of the difference between the distribution of methylation fraction values determined for a sample from a subject and the distribution of methylation fraction values in samples from a plurality of healthy individuals or diseased individuals.
126. The method of clause 125, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
127. The method of clause 125 or clause 126, wherein the genomic profile for the subject further comprises results from a nucleic acid sequencing-based test.
128. The method of any one of clauses 125 to 127, further comprising selecting an anti-cancer therapy, administering an anti-cancer therapy, or applying an anti-cancer therapy to the subject based on the generated genomic profile.
129. The method of any one of clauses 34 to 69, wherein the determination of determination of the difference between the distribution of methylation fraction values determined for a sample from a subject and the distribution of methylation fraction values in samples from a plurality of healthy individuals or diseased individuals is used in making suggested treatment decisions for the subject.
130. The method of any one of clauses 34 to 69, wherein the determination of determination of the difference between the distribution of methylation fraction values determined for a sample from a subject and the distribution of methylation fraction values in samples from a plurality of healthy individuals or diseased individuals is used in applying or administering a treatment to the subject.
131. A system comprising:
   one or more processors; and
   a memory communicatively coupled to the one or more processors and configured to store instructions that, when executed by the one or more processors, cause the system to perform the method of any one of clauses 34 to 108.
132. A non-transitory computer-readable storage medium storing one or more programs, the one or more programs comprising instructions, which when executed by one or more processors of a system, cause the system to perform the method of any one of clauses 34 to 108.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, sequence read data for the plurality of sequence reads;
determining, using the one or more processors, a methylation fraction value for a plurality of CpG sites within each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and
performing, using the one or more processors, a statistical analysis to compare a distribution of the methylation fraction values determined for a plurality of CpG sites within one or more genomic regions of the set of selected genomic regions in the subject to a corresponding distribution of methylation fraction values determined for the plurality of CpG sites within one or more genomic regions of the set of selected genomic regions for the plurality of healthy individuals,
wherein a difference between the distribution of methylation fraction values determined for the subject and the distribution of methylation fraction values determined for the plurality of healthy individuals is indicative of a presence of disease in the subject.

2. The method of claim 1, further comprising comparing, using the one or more processors, the difference between the distribution of methylation fraction values determined for the subject and the distribution of methylation fraction values determined for the plurality of healthy individuals to a first predetermined threshold.

3. The method of claim 2, wherein: (i) if the difference is greater than or equal to the first predetermined threshold, a disease-positive status is output by the one or more processors, or (ii) if the difference is less than the first predetermined threshold, a disease-negative status is output by the one or more processors.

4. (canceled)

5. (canceled)

6. (canceled)

7. The method of claim 1, further comprising use of a Kullbeck-Liebler divergence or informatics entropy-based approach to evaluate a difference between the distribution of methylation fraction values determined for the subject and the distribution of methylation fraction values determined for the plurality of healthy individuals.

8. (canceled)

9. A method for detecting disease in a subject, the method comprising:
receiving, at one or more processors, sequence read data for a plurality of sequence reads derived from a sample from the subject;
determining, using the one or more processors, a fragment-level methylation status value for each genomic region of a set of selected genomic regions based on the sequence read data, wherein each selected genomic region of the set comprises a plurality of CpG sites that are proximal to each other and that exhibit a consistent methylation status in sequence read data for a plurality of healthy individuals; and
performing, using the one or more processors, a statistical analysis to compare the fragment-level methylation status values determined for one or more genomic regions of the set of selected genomic regions in the subject to corresponding fragment-level methylation status values determined for one or more genomic regions of the set of selected genomic regions for the plurality of healthy individuals,
wherein a difference between the fragment-level methylation fraction status values determined for one or more genomic regions for the subject and the fragment-level methylation fraction status values determined for the corresponding one or more genomic regions for the plurality of healthy individuals is indicative of a presence of disease in the subject.

10. The method of claim 9, further comprising comparing, using the one or more processors, the difference between the fragment-level methylation status values determined for the one or more genomic regions for the subject and the fragment-level methylation fraction status values determined for the corresponding one or more genomic regions for the plurality of healthy individuals to a second predetermined threshold.

11. The method of claim 10, wherein: (i) if the difference is greater than or equal to the second predetermined threshold, a disease-positive status is output by the one or more processors, or (ii) if the difference is less than the second predetermined threshold, a disease-negative status is output by the one or more processors.

12. (canceled)

13. The method of claim 9, wherein the difference between the fragment-level methylation fraction status values determined for one or more genomic regions for the subject and the fragment-level methylation fraction status values determined for the corresponding one or more genomic regions for the plurality of healthy individuals comprises a difference in a mean, median, mode, 10^{th} percentile, 25^{th} percentile, 75^{th} percentile, or 90^{th} percentile of the fragment-level methylation status values.

14. (canceled)

15. The method of claim 1, wherein the set of selected genomic regions is selected based on the subject's age, sex, race, body mass index, smoking history, clinical history, family history, genetic predisposition for disease, diagnosed disease, polygenic risk score, or any combination thereof.

16. (canceled)

17. (canceled)

18. (canceled)

19. The method of claim 1, wherein the methylation fraction values or fragment-level methylation fraction status values determined for the subject are used as input for a machine learning model configured to output a prediction of a probability that the subject has the disease.

20. (canceled)

21. The method of claim 1, wherein the methylation fraction value for each CpG site or the fragment-level methylation status value for each genomic region is determined based on sequence read data obtained using a targeted nucleic acid sequencing method that utilizes proteins comprising methyl-binding domains to capture genomic regions comprising methylated CpG sites.

22. The method of claim 1, wherein the disease is cancer.

23. A method for identifying informative genomic sub-regions, the method comprising:
receiving, at one or more processors, sequence read data for a plurality of candidate genomic regions in samples from a plurality of healthy individuals;
determining, using the one or more processors, a methylation fraction value for a plurality of CpG sites within each of the plurality of candidate genomic regions in each of the plurality of samples from the healthy individuals based on the sequence read data;
determining, using the one or more processors, a fitted methylation fraction value for a plurality of CpG sites within each candidate genomic region by fitting genomic positions of the CpG sites and corresponding methylation fraction values of the plurality of CpG sites to a local regression model;
identifying, using the one or more processors, one or more genomic sub-regions for each candidate genomic region by iteratively evaluating sequences of different length that: (i) exhibit a consistent methylation status in sequence read data for a plurality of healthy individuals, and (ii) comprise a plurality of CpG sites that are proximal to each other;
identifying starting and ending genomic positions for each of the one or more genomic sub-regions; and
assigning a label to each CpG site within the one or more genomic sub-regions based on their position within the genomic sub-region.

24. The method of claim 23, wherein identifying the one or more genomic sub-regions for each candidate genomic region comprises repeating, for each successive CpG site starting at a 5′-end of each candidate genomic region, the steps of:
comparing a genomic position and a fitted methylation fraction value for a current CpG site to a genomic position and fitted methylation fraction value for a next CpG site;
determining a separation distance between the current CpG site and the next CpG site;
determining a methylation metric calculated based on the fitted methylation fraction values for a current genomic sub-region and
in response to the separation distance being greater than a specified number of bases, d: (1) assigning the next CpG site to a new genomic sub-region, and (2) terminating a current genomic sub-region; or
in response to the methylation metric being greater than a predetermined value, M, if the next CpG site were included in the current genomic sub-region: (1) assigning the next CpG site to a new genomic sub-region, and (2) terminating the current genomic sub-region; or
assigning the next CpG site to the current genomic sub-region.

25. The method of claim 23, further comprising assessing methylation fraction value data for the one or more identified genomic sub-regions for samples from a plurality of patients diagnosed with a specified disease to identify genomic sub-regions that may be used to differentiate between patients diagnosed with the disease and healthy individuals.

26. The method of claim 23, wherein the plurality of candidate genomic regions are selected from: (i) annotated genomic regions identified in a genomics database as exhibiting differential methylation status for a specified disease, (ii) genomic regions identified in a scientific publication as exhibiting differential methylation status for a specified disease, (iii) genomic regions identified in a genomics database that comprise cell type-specific markers, markers related to transcriptional programs, genes, or additional genomic features that have not been shown to exhibit differential methylation in a specified disease or (iv) genomic regions associated with a functional pathway in a specified disease.

27. (canceled)

28. (canceled)

29. (canceled)

30. The method of claim 26, wherein the specified disease is cancer and the genomic regions comprise tumor suppressor genes or oncogenes.

31. The method of claim 26, wherein the specified disease is an immune system disorder and the genomic regions comprise major histocompatibility complex genes.

32. The method of claim 23, wherein the identified genomic sub-regions are further extended by a short section of additional nucleic acid sequence at either end of the genomic sub-region defined by a corresponding pair of “start” and “end” CpGs.

33. (canceled)

34. The method of claim 1, wherein the determination of a difference between the distribution of methylation fraction values determined for the subject and the distribution of methylation fraction values determined for a plurality of healthy individuals or diseased individuals is used to diagnose or confirm a diagnosis of cancer in the subject.
